(19) United States
(12) Patent Application Publication
(10) Pub. No.: US 2024/0408244 A1
(43) Pub. Date: Dec. 12, 2024

(54) COMPOSITIONS AND METHODS FOR CHARACTERIZING AND TREATING NEURODEGENERATIVE DISORDERS

(71) Applicants: Arizona Board of Regents on Behalf of the University of Arizona, Tucson, AZ (US); Duke University, Durham, NC (US)

(72) Inventors: Rui Chang, Tucson, AZ (US); Roberta Diaz Brinton, Tucson, AZ (US); Rima Kaddurah-Daouk, Durham, NC (US)

(21) Appl. No.: 18/702,693

(22) PCT Filed: Oct. 21, 2022
(86) PCT No.: PCT/US22/47359

§ 371 Date: Apr. 18, 2024

Related U.S. Application Data
(60) Provisional application No. 63/270,237, filed on Oct. 21, 2021.

(51) Int. Cl. A61K 49/00; A61P 25/28; G01N 33/68; G01N 33/92

(52) U.S. Cl. CPC A61K 49/0004; A61P 25/28; G01N 33/6896; G01N 33/92; G01N 2800/2821

(57) ABSTRACT

Provided herein are compositions and methods for diagnosing, prognosing, and treating neurodegenerative diseases in a male and female subject at risk of or diagnosed with a neurodegenerative disease. In particular, provided herein are customized metabolite analyses for characterizing and treating neurodegenerative diseases in specific subject groups from a sample from said subject.

### CROSS-REFERENCE TO RELATED APPLICATION

The present application claims priority to U.S. Provisional Patent Application No. 63/270,237, filed Oct. 21, 2021, which is hereby incorporated by reference in its entirety.

### STATEMENT REGARDING FEDERALLY SPONSORED RESEARCH OR DEVELOPMENT

This invention was made with government support under Grant No. AG057457 and AG059093, awarded by National Institutes of Health. The government has certain rights in the invention.

### FIELD

Provided herein are compositions and methods for diagnosing, prognosing, and treating neurodegenerative diseases. In particular, provided herein are customized metabolite analyses for characterizing and treating neurodegenerative diseases in specific subject groups.

### BACKGROUND

There is an urgent need to develop novel therapies for neurodegenerative diseases and conditions such as Alzheimer's disease (AD). 10% of persons over age 65 and up to 50% over age 85 have dementia, with over 30 million people affected worldwide. AD affects over 26 million people worldwide and currently there is no cure for the disease. With the growing number of people living to older ages, there is an urgency to better understand elements of the pathogenic pathway, discover agents that target these elements, and establish their roles in the treatment and prevention of AD.

As such, improved methods for treating neurodegenerative disorders (e.g., AD) are needed.

The present invention addresses this need.

### SUMMARY

Although a significant amount of research has focused on the role of metabolism in the pathophysiology of late onset Alzheimer's disease, the effects of major AD risk factors, including sex and APOE genotype, on the associations between blood-based metabolites and AD diagnosis and pathology is still understudied. One major goal of Accelerating Medicines Partnership-Alzheimer's Disease (AMP-AD) consortium at National Institute of Aging is to develop accurate and robust blood-based metabolic biomarkers for AD diagnosis that is less invasive and more affordable and accessible in clinic. Experiments described herein employed computational systems biology approach to build sex and APOE genotype-specific metabolic networks of 127 metabolic measurements derived from 656 serum samples (294 AD and 362 CN) in individuals from the AD neuroimaging initiative (ADNI) cohort with clinical AD and cognitively normal controls. Based on these network structures, patient group-specific (sex and APOE genotype-specific) blood-based metabolic biomarkers and key metabolic mediators associated with AD were identified. These metabolic biomarkers were associated with AD diagnosis, and cognitive declines (ADAS-Cog total score, memory, and executive function) in each patient group.

For example, in some embodiments, provided herein is a method of measuring a panel of biomarkers in a male subject at risk of or diagnosed with a neurodegenerative disease, comprising: measuring the level of one or more (e.g., 1, 2, 3, 4, 5, 6, 7, 8, 9, 10, 11, 12, 13, 14, 15, 16, 17, or all) metabolites selected from, for example, C18, PC aa C42:6, lysoPC a C18:0, PC ae C36:4, Kynurenine, PC ae C40:2, PC ae C42:5, PC ae, C36:5, lysoPC a C20:3, Cit, lysoPC a C16:0, SM C24:0, C16, PC ae C42:3, PC aa C32:1, Ile, C4, or Creatinine in a sample from the subject.

Also provided is a method of measuring a panel of biomarkers in a female subject at risk of or diagnosed with a neurodegenerative disease, comprising: measuring the level of one or more (e.g., 1, 2, 3, 4, 5, 6, 7, 8, 9, 10, or all) metabolites selected from, for example, PC ae C40:2, SM C16:0, C14:1, alpha-AAA, C4, Trp, Ile, SM C26:0, SM C16:1, PC aa C36:5, or Thr in a sample from the subject.

Other embodiments provide a method of measuring a panel of biomarkers in a APOE e4⁺ subject at risk of or diagnosed with a neurodegenerative disease, comprising: measuring the level of one or more (e.g., 1, 2, 3, 4, 5, 6, 7, 8, 9, 10, 11, 12, 13, 14, 15, 16, 17, 18, or all) metabolites selected from, for example, PC ae C36:0, PC ae C42:5, PC aa C32:0, PC aa C42:6, PC ae C36:5, lysoPC a C16:0, PC aa C36:0, PC aa C32:3, PC ae C34:2, C16:1, lysoPC a C18:0, SM C18:1, PC aa C36:1, PC aa C34:4, PC ae, C36:4, C18:1, PC aa C42:5, SM (OH) C24:1, or PC aa C38:0 in a sample from the subject.

Further embodiments provide a method of measuring a panel of biomarkers in a APOE e4″ subject at risk of or diagnosed with a neurodegenerative disease, comprising: measuring the level of one or more (e.g., 1, 2, 3, 4, 5, 6, 7, 8, 9, 10, 11, 12, 13, 14, 15, 16, 17, 18, 19, 20, 21, 22, 23, 24, or all) metabolites selected from, for example, C12, PC aa C36:5, Thr, SM C16:1, SM (OH) C24:1, PC ae C42:4, Ala, PC ae C36:5, Creatinine, PC ae C36:0, Cit, SM C20:2, PC aa C42:0, PC ae C44:3, lysoPC a C16:1, PC aa C40:6, C14:1, C4, PC aa C38:6, lysoPC a C18:1, PC aa C32:1, PC aa C38:0, PC aa C36:6, PC ae C42:2 or PC ae C40:6 in a sample from the subject.

Additional embodiments provide a method of measuring a panel of biomarkers in a male APOE e4⁺ subject at risk of or diagnosed with a neurodegenerative disease, comprising: measuring the level of one or more (e.g., 1, 2, 3, 4, 5, 6, 7, 8, or all) metabolites selected from, for example Taurine, PC ae C36:5, PC aa C42:6, PC aa C38:4, SM (OH) C22:1, PC ae C32:1, PC ae C32:2, lysoPC a C18:0, or Asn in a sample from the subject.

Yet other embodiments provide a method of measuring a panel of biomarkers in a female APOE e4⁺ subject at risk of or diagnosed with a neurodegenerative disease, comprising: measuring the level of one or more (e.g., 1, 2, 3, 4, or all) metabolites selected from, for example, PC aa C34:4, PC ae C40:2, lysoPC a C18:1, PC aa C38:3, or PC aa C42:5 in a sample from the subject.

In certain embodiments, provided herein is a method of measuring a panel of biomarkers in a male APOE e4⁻ subject at risk of or diagnosed with a neurodegenerative disease, comprising: measuring the level of one or more (e.g., 1, 2, 3, 4, 5, 6, 7, 8, 9, 10, or all) metabolites selected from, for example, C14:1-OH, Ala, PC ae C40:4, PC ae C36:0, SM (OH) C24:1, PC ae C40:2, PC ae C38:5, Tyr, Sarcosine, lysoPC a C16:0, or C9 in a sample from the subject.

In further embodiments, provided herein is a method of measuring a panel of biomarkers in a female APOE e4⁻ subject at risk of or diagnosed with a neurodegenerative disease, comprising: measuring the level of one or more (e.g., 1, 2, 3, 4, 5, 6, 7, 8, 9, 10, 11, 12, 13, 14, 15, 16, or all) metabolites selected from, for example, C16:1, SM C16:0, C12, C9, PC ae C36:4, Lys, PC ae C32:2, PC ae C44:6, PC aa C36:5, SM (OH) C14:1, PC aa C34:3, Creatinine, PC ae C36:0, PC aa C28:1, SM C26:0, PC ae C40:3, or SM (OH) C22:2 in a sample from the subject.

The present disclosure is not limited to particular neurodegenerative diseases. Examples include but are not limited to, Alzheimer's disease (AD).

The present disclosure is not limited to particular sample types. Examples include but are not limited to, blood and blood products (e.g., serum).

Certain aspects of the disclosure relate to screening (e.g., drug screening) embodiments. For example, in some embodiments, the method further comprises administering a test compound to the subject prior to the measuring. In some embodiments, the test compound is a treatment for the neurodegenerative disease (e.g., a pharmaceutical agent or a nutraceutical (e.g., comprising one or more amino acids and/or lipids)). In some embodiments, the method is repeated one or more times (e.g., monthly, yearly, or at another interval).

In some specific embodiments, the presence of the markers is indicative of a diagnosis of a neurodegenerative disease or a prognosis (e.g., rate of cognitive decline) for a neurodegenerative disease.

Also provide is a method of treating or preventing neurodegenerative disease in a subject, comprising: administering a composition comprising one or more compounds selected from those in tables 10-15 to a subject in need thereof or the use of a composition comprising one or more compounds selected from those in tables 10-15 to treat or prevent a neurodegenerative disease in a subject.

Additional embodiments are described herein.

### BRIEF DESCRIPTION OF THE DRAWINGS

FIG. 1. Workflow of Metabolic Analysis in ADNI.

FIG. 2. Sex and APOE-specific Consensus Predictive Metabolic Network The robust causal predictive network model was built to identify the upstream metabolites and pathways associated with AD in male (A), female (B), APOE ε4+ (C), APOE ε4− (D), male APOE ε4+ (E), male APOE ε4− (F), female APOE ε4+ (G), female APOE ε4− (H), overall 656 AD and CN samples (I).

FIG. 3. Sex and APOE-specific AD-associated metabolic heterogeneity.

FIG. 4. Sex- and APOE-specific Metabolic Differential Expression Analysis. (A) Male vs Female; (B) APOE ε4+ vs APOE ε4−; (C) Male APOE ε4+ vs Male APOE ε4−; (D) Male APOE ε4—vs Female APOE ε4+; (E) Female APOE ε4+ vs Female APOE ε4−; (F) Male APOE ε4+ vs Female APOE ε4+; (G) Male APOE ε4—vs Female APOE ε4+; (H) Male APOE ε4+ vs Female APOE ε4−.

FIG. 5. Biomarker panel for AD diagnosis. All: all metabolites in the data; DE only: biomarkers derived from significant DE metabolites; NetworkOnly: biomarkers derived from key driver metabolites in the network; DE&Network: biomarkers derived from the combination of significant DE and key driver metabolites; Network&Clinic: biomarkers derived from key driver metabolites in the network and age, BMI and/or education; DE&Network&Clinic: biomarkers derived from combination of significant DE metabolites, key driver metabolites and age, BMI, and/or education.

FIG. 6. Biomarker panel association with clinic assessment and cognition decline. The association of biomarker panel (A: biomarkers derived from combination key driver metabolites and age, BMI and/or education; B: biomarkers derived from key driver metabolites) with diagnosis (Dx) and clinical assessments (ADAS-Cog 13 Score, ADAS-Cog Total Score, memory function and executive function).

### DEFINITIONS

Although any methods and materials similar or equivalent to those described herein can be used in the practice or testing of embodiments described herein, some preferred methods, compositions, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the terms “comprise”, “include”, and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

The term “diagnostic assay” can be used interchangeably with “diagnostic method” and refers to the detection of the presence or nature of a pathologic condition.

As used herein, the terms “prevent,” “prevention,” and preventing” may refer to reducing the likelihood of a particular condition or disease state (e.g., neurodegenerative disease such as AD) from occurring in a subject not presently experiencing or afflicted with the condition or disease state. The terms do not necessarily indicate complete or absolute prevention. The terms may also refer to delaying the onset of a particular condition or disease state in a subject not presently experiencing or afflicted with the condition or disease state. “Prevention,” encompasses any administration or application of a therapeutic or technique to reduce the likelihood or delay the onset of a disease developing (e.g., in a mammal, including a human). Such a likelihood may be assessed for a population or for an individual.

As used herein, the terms “treat,” “treatment,” and “treating” refer to reducing the amount or severity of a particular condition, disease state (e.g., AD), or symptoms thereof, in a subject presently experiencing or afflicted with the condition or disease state. The terms do not necessarily indicate complete treatment (e.g., total elimination of the condition, disease, or symptoms thereof). “Treatment,” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process.

As used herein, the term “subject” as used herein includes all members of the animal kingdom including mammals, and suitably refers to humans. Optionally, the term “subject” includes mammals that have been diagnosed with a neurodegenerative disorder or are at increased risk of developing such a disorder.

As used herein, the word “presence” or “absence” (or, alternatively, “present or “absent”) is used in a relative sense to describe the amount or level of a particular entity (e.g., an analyte). For example, when an analyte is said to be “present” in a test sample, it means the level or amount of this analyte is above a pre-determined threshold; conversely, when an analyte is said to be “absent” in a test sample, it means the level or amount of this analyte is below a pre-determined threshold. The pre-determined threshold may be the threshold for detectability associated with the particular test used to detect the analyte or any other threshold. When an analyte is “detected” in a sample it is “present” in the sample; when an analyte is “not detected” it is “absent” from the sample. Further, a sample in which an analyte is “detected” or in which the analyte is “present” is a sample that is “positive” for the analyte. A sample in which an analyte is “not detected” or in which the analyte is “absent” is a sample that is “negative” for the analyte.

As used herein, an “increase” or a “decrease” refers to a detectable (e.g., measured) positive or negative change, respectively, in the value of a variable relative to a previously measured value of the variable, relative to a pre-established value, and/or relative to a value of a standard control. An increase is a positive change preferably at least 10%, more preferably 50%, still more preferably 2-fold, even more preferably at least 5-fold, and most preferably at least 10-fold relative to the previously measured value of the variable, the pre-established value, and/or the value of a standard control. Similarly, a decrease is a negative change preferably at least 10%, more preferably 50%, still more preferably at least 80%, and most preferably at least 90% of the previously measured value of the variable, the pre-established value, and/or the value of a standard control. Other terms indicating quantitative changes or differences, such as “more” or “less,” are used herein in the same fashion as described above.

### DETAILED DESCRIPTION

Alzheimer's disease (AD) is characterized by the accumulation of amyloid plaques and phosphorylated tau tangles in the brain as pathologic hallmarks. In recent years, an increasing number of studies have discovered novel molecular mechanisms independent of Aβ pathway that are associated with AD pathology, including impaired calcium and lipid homeostasis, mitochondrial dysfunction, altered synaptic transmission, oxidative stress, and inflammation [1-5]. These molecular mechanisms mediated the effects of genetics and their interactions with various risk factors, such as age, sex, and APOE genotype (Letenneur, L., et al., Education and the risk for Alzheimer's disease: sex makes a difference. EURODEM pooled analyses. EURODEM Incidence Research Group. Am J Epidemiol, 2000. 151 (11): p. 1064-71 (Dubal, D. B., Sex difference in Alzheimer's disease: An updated, balanced and emerging perspective on differing vulnerabilities. Handb Clin Neurol, 2020. 175: p. 261-273; Swanwick, G. and B. A. Lawlor, Is female gender a risk factor for Alzheimer's disease? Int Psychogeriatr, 1999. 11 (3): p. 219-22; Sando, S. B., et al., APOE epsilon 4 lowers age at onset and is a high risk factor for Alzheimer's disease; a case control study from central Norway. BMC Neurol, 2008. 8: p. 9; Ishii, M. and C. Iadecola, Risk factor for Alzheimer's disease breaks the blood-brain barrier. Nature, 2020. 581 (7806): p. 31-32)), affecting the onset and progression of AD.

Among the AD-associated risk factors, older age, female sex and APOE ε4 genotype have been reported to play significant roles in metabolism (Gamache, J., Y. Yun, and O. Chiba-Falek, Sex-dependent effect of APOE on Alzheimer's disease and other age-related neurodegenerative disorders. Dis Model Mech, 2020. 13 (8); Shang, Y., et al., Evidence in support of chromosomal sex influencing plasma based metabolome vs APOE genotype influencing brain metabolome profile in humanized APOE male and female mice. PLOS One, 2020. 15 (1): p. e0225392; Christensen, A. and C. J. Pike, APOE genotype affects metabolic and Alzheimer-related outcomes induced by Western diet in female EFAD mice. FASEB J, 2019. 33 (3): p. 4054-4066; Arnold, M., et al., Sex and APOE epsilon4 genotype modify the Alzheimer's disease serum metabolome. Nat Commun, 2020. 11 (1): p. 1148; Riedel, B. C., P. M. Thompson, and R. D. Brinton, Age, APOE and sex: Triad of risk of Alzheimer's disease. J Steroid Biochem Mol Biol, 2016. 160: p. 134-47). Metabolomics measures biochemical products of genetic, transcriptomic, and proteomic reactions and has great promises to capture cellular and disease state across complex physiological and pathological pathways driven by multiscale biological network between genetic and environmental risk factors (Wilkins, J. M. and E. Trushina, Application of Metabolomics in Alzheimer's Disease. Front Neurol, 2017. 8: p. 719; Trushina, E. and M. M. Mielke, Recent advances in the application of metabolomics to Alzheimer's Disease. Biochim Biophys Acta, 2014. 1842 (8): p. 1232-9; Cuperlovic-Culf, M. and A. Badhwar, Recent advances from metabolomics and lipidomics application in alzheimer's disease inspiring drug discovery. Expert Opin Drug Discov, 2020. 15 (3): p. 319-331). In AD, metabolomics has been increasingly applied to understand how various genetic alterations drive disease progression from preclinical and prodromal stage to irreversible neurodegenerative disorder through impacts on metabolism (Wilkins et al, supra; Trushina et al., supra; Cuperlovic-Culf et al., supra; Hunsberger, H. C., et al., Author Correction: Divergence in the metabolome between natural aging and Alzheimer's disease. Sci Rep, 2020. 10 (1): p. 19863; Niedzwiecki, M. M., et al., High-resolution metabolomic profiling of Alzheimer's disease in plasma. Ann Clin Transl Neurol, 2020. 7 (1): p. 36-45). For instance, aging is the most significant regulator of mitochondrial function and energy metabolism (Sun, N., R. J. Youle, and T. Finkel, The Mitochondrial Basis of Aging. Mol Cell, 2016. 61 (5): p. 654-666; Horan, M. P., N. Pichaud, and J. W. Ballard, Review: quantifying mitochondrial dysfunction in complex diseases of aging. J Gerontol A Biol Sci Med Sci, 2012. 67 (10): p. 1022-35; Slack, C., Ras signaling in aging and metabolic regulation. Nutr Healthy Aging, 2017. 4 (3): p. 195-205; Finkel, T., The metabolic regulation of aging. Nat Med, 2015. 21 (12): p. 1416-23). A decline in mitochondrial function plays a key role in the aging process and increases the incidence of many age-related disorders including AD (Horan et a., supra; Srivastava, S., The Mitochondrial Basis of Aging and Age-Related Disorders. Genes (Basel), 2017. 8 (12); Salminen, A., et al., Mitochondrial dysfunction and oxidative stress activate inflammasomes: impact on the aging process and age-related diseases. Cell Mol Life Sci, 2012. 69 (18): p. 2999-3013; Haas, R. H., Mitochondrial Dysfunction in Aging and Diseases of Aging. Biology (Basel), 2019. 8 (2); Hamrick, M. W. and A. M. Stranahan, Metabolic regulation of aging and age-related disease. Ageing Res Rev, 2020: p. 101175; Duan, W., Sirtuins: from metabolic regulation to brain aging. Front Aging Neurosci, 2013. 5: p. 36). Recent studies demonstrated aging is associated with changes in levels of phosphatidylcholines (PC), sphingomyelins (SM), acyl-carnitines, ceramides, and amino acids (Yu, Z., et al., Human serum metabolic profiles are age dependent. Aging Cell, 2012. 11 (6): p. 960-7; Gonzalez-Covarrubias, V., et al., Lipidomics of familial longevity. Aging Cell, 2013. 12 (3): p. 426-34). APOE genotype is associated with alterations in cholesterol metabolism and lipid homeostasis (Teslovich, T. M., et al., Biological, clinical and population relevance of 95 loci for blood lipids. Nature, 2010. 466 (7307): p. 707-13; Tudorache, I. F., V. G. Trusca, and A. V. Gafencu, Apolipoprotein E-A Multifunctional Protein with Implications in Various Pathologies as a Result of Its Structural Features. Comput Struct Biotechnol J, 2017. 15: p. 359-365). Genome-wide association studies (GWAS) identified common genetic variants in APOE associated with blood cholesterol levels (Shin, S. Y., et al., An atlas of genetic influences on human blood metabolites. Nat Genet, 2014. 46 (6): p. 543-550; Rasmussen-Torvik, L. J., et al., High density GWAS for LDL cholesterol in African Americans using electronic medical records reveals a strong protective variant in APOE. Clin Transl Sci, 2012. 5 (5): p. 394-9) and SM levels (Long, T., et al., Whole-genome sequencing identifies common-to-rare variants associated with human blood metabolites. Nat Genet, 2017. 49 (4): p. 568-578; Mielke, M. M., et al., The Association Between Plasma Ceramides and Sphingomyelins and Risk of Alzheimer's Disease Differs by Sex and APOE in the Baltimore Longitudinal Study of Aging. J Alzheimers Dis, 2017. 60 (3): p. 819-828). Female sex is a well-known major risk factor for AD. In the United States, it is assumed that more than 60% of all individuals who were diagnosed with AD at age 65 or older, are women and the risk of developing AD at age 45 is estimated to be almost double in female than in male (Zhao, N., et al., Alzheimer's Risk Factors Age, APOE Genotype, and Sex Drive Distinct Molecular Pathways. Neuron, 2020. 106 (5): p. 727-742 e6; Underwood, E. A., et al., Sex Differences in Depression as a Risk Factor for Alzheimer's Disease: A Systematic Review. Innov Aging, 2019. 3 (2): p. igz015; Uchoa, M. F., V. A. Moser, and C. J. Pike, Interactions between inflammation, sex steroids, and Alzheimer's disease risk factors. Front Neuroendocrinol, 2016. 43: p. 60-82; Tariman, J. D., Men and Alzheimer's disease. Sex differences in risk. Adv Nurse Pract, 2009. 17 (2): p. 23; Scheyer, O., et al., Female Sex and Alzheimer's Risk: The Menopause Connection. J Prev Alzheimers Dis, 2018. 5 (4): p. 225-230; Munro, C. A., Sex differences in Alzheimer's disease risk: are we looking at the wrong hormones? Int Psychogeriatr, 2014. 26 (10): p. 1579-84; Lai, F., et al., Sex differences in risk of Alzheimer's disease in adults with Down syndrome. Alzheimers Dement (Amst), 2020. 12 (1): p. e12084; de Lange, A. G., et al., Women's brain aging: Effects of sex-hormone exposure, pregnancies, and genetic risk for Alzheimer's disease. Hum Brain Mapp, 2020; Beam, C. R., et al., A Twin Study of Sex Differences in Genetic Risk for All Dementia, Alzheimer's Disease (AD), and Non-AD Dementia. J Alzheimers Dis, 2020. 76 (2): p. 539-551). Increasing number of evidences suggested that metabolic homeostasis is differentially regulated between men and women (Sugiyama, M. G. and L. B. Agellon, Sex differences in lipid metabolism and metabolic disease risk. Biochem Cell Biol, 2012. 90 (2): p. 124-41; Roeters van Lennep, J. E., et al., Risk factors for coronary heart disease: implications of gender. Cardiovasc Res, 2002. 53 (3): p. 538-49; Mauvais-Jarvis, F., Sex differences in metabolic homeostasis, diabetes, and obesity. Biol Sex Differ, 2015. 6: p. 14; Murphy, M. O. and A. S. Loria, Sex-specific effects of stress on metabolic and cardiovascular disease: are women at higher risk? Am J Physiol Regul Integr Comp Physiol, 2017. 313 (1): p. R1-R9) due to reversible hormonal effects, irreversible developmental processes, and gene expression differences from the X and Y-chromosome (Chella Krishnan, K., M. Mehrabian, and A. J. Lusis, Sex differences in metabolism and cardiometabolic disorders. Curr Opin Lipidol, 2018. 29 (5): p. 404-410). In a recent study comparing blood-based metabolic difference in healthy older women and men, women showed higher levels of most lipids, except lyso-PCs, while males showed higher levels of most amino acids including branched chain amino acids (BCAAs) except glycine and serine (Mittelstrass, K., et al., Discovery of sexual dimorphisms in metabolic and genetic biomarkers. PLOS Genet, 2011. 7 (8): p. e1002215; Krumsiek, J., et al., Gender-specific pathway differences in the human serum metabolome. Metabolomics, 2015. 11 (6): p. 1815-1833). In longevity studies, higher levels of lipids (i.e. PCs and SMs) were associated with longevity in women but not in men (Gonzalez-Covarrubias, V., et al., supra; Johnson, A. A. and A. Stolzing, The role of lipid metabolism in aging, lifespan regulation, and age-related disease. Aging Cell, 2019. 18 (6): p. e13048; Willmes, D. M., et al., The longevity gene INDY (I'm Not Dead Yet) in metabolic control: Potential as pharmacological target. Pharmacol Ther, 2018. 185: p. 1-11; Lopez-Otin, C., et al., Metabolic Control of Longevity. Cell, 2016. 166 (4): p. 802-821; Goyal, M. S., et al., Persistent metabolic youth in the aging female brain. Proc Natl Acad Sci USA, 2019. 116 (8): p. 3251-3255; Austad, S. N. and K. E. Fischer, Sex Differences in Lifespan. Cell Metab, 2016. 23 (6): p. 1022-1033).

Despite tremendous efforts to develop less invasive, more affordable and robust blood-based metabolic biomarkers to diagnose AD over the past decades (Greenberg, N., et al., A proposed metabolic strategy for monitoring disease progression in Alzheimer's disease. Electrophoresis, 2009. 30 (7): p. 1235-9; Thambisetty, M. and S. Lovestone, Blood-based biomarkers of Alzheimer's disease: challenging but feasible. Biomark Med, 2010. 4 (1): p. 65-79; Bekris, L. M. and J. B. Leverenz, Emerging blood-based biomarkers for Alzheimer disease. Cleve Clin J Med, 2020. 87 (9): p. 537-539; de Leeuw, F. A., et al., Blood-based metabolic signatures in Alzheimer's disease. Alzheimers Dement (Amst), 2017. 8: p. 196-207), current clinic diagnosis of AD still uses invasive cerebrospinal fluid (CSF) or expensive positron emission tomography (PET) to detect amyloid β (Aβ) and tau proteins, their prohibitive cost, insufficient accessibility, and invasiveness limit their usage to monitor the cognitive decline and detect early changes of neuropathology and pathophysiology associated with AD. In fact, a major goal of AMP-AD and ADMC consortium is to develop such accurate and robust blood-based metabolic biomarker panels for AD diagnosis. Current efforts on development of metabolic biomarkers by pooling patients with distinct metabolic mechanisms convoluted with sex and APOE genotype may explain the inconsistent findings among these existing studies.

To address the challenges in understanding the metabolic impacts of sex and APOE genotype in AD, experiments described herein employed a computational systems biology approach to reconstruct sex and APOE genotype-specific metabolic networks from serum-based metabolites of the individuals in AD neuroimaging initiative (ADNI) cohort. Based on these network structures, patient group-specific (stratified by sex and APOE genotype) metabolic biomarkers in the blood for AD diagnosis and treatment were identified. These biomarkers not only are predictive of clinical diagnosis but also associated with AD pathology (CSF Aβ 1-42 and CSF p181-Tau) and cognitive decline (Total-13, ADNI-MEM, ADNI-EF) in each patient group.

The markers described herein are unique to a given patient population (See e.g., Tables 3-10) and provide for customized research, screening, diagnostic, and therapeutic uses.

Metabolites may be detected using any suitable method including, but not limited to, liquid and gas phase chromatography, alone or coupled to mass spectrometry (See e.g., experimental section below), NMR (See e.g., US patent publication 20070055456, herein incorporated by reference), immunoassays, chemical assays, spectroscopy and the like. In some embodiments, commercial systems for chromatography and NMR analysis are utilized.

In other embodiments, metabolites (e.g., biomarkers and derivatives thereof) are detected using optical imaging techniques such as magnetic resonance spectroscopy (MRS), magnetic resonance imaging (MRI), CAT scans, ultra sound, MS-based tissue imaging or X-ray detection methods (e.g., energy dispersive x-ray fluorescence detection).

Any suitable method may be used to analyze the biological sample in order to determine the presence, absence or level(s) of the one or more metabolites in the sample. Suitable methods include chromatography (e.g., HPLC, gas chromatography, liquid chromatography), mass spectrometry (e.g., MS, MS-MS), enzyme-linked immunosorbent assay (ELISA), antibody linkage, other immunochemical techniques, biochemical or enzymatic reactions or assays, and combinations thereof. Further, the level(s) of the one or more metabolites may be measured indirectly, for example, by using an assay that measures the level of a compound (or compounds) that correlates with the level of the biomarker(s) that are desired to be measured.

The levels of one or more of the recited metabolites may be determined in the methods of the present invention. For example, the level(s) of one metabolites, two or more metabolites, three or more metabolites, four or more metabolites, five or more metabolites, six or more metabolites, seven or more metabolites, eight or more metabolites, nine or more metabolites, ten or more metabolites, etc., including a combination of some or all of the metabolites described herein may be determined and used in such methods. Determining levels of combinations of the metabolites may allow greater sensitivity and specificity in the methods, such as diagnosing AD and aiding in the diagnosis of AD. For example, ratios of the levels of certain metabolites in biological samples may allow greater sensitivity and specificity in diagnosing AD and allow better differentiation or characterization of AD.

In some embodiments, as described herein, specific marker are selected based on the gender and/or APOE e4 status of the subject. For example, in some embodiments, provided herein is a method of measuring a panel of biomarkers in a male subject at risk of or diagnosed with a neurodegenerative disease, comprising: measuring the level of one or more (e.g., 1, 2, 3, 4, 5, 6, 7, 8, 9, 10, 11, 12, 13, 14, 15, 16, 17, or all) metabolites selected from, for example, C18, PC aa C42:6, lysoPC a C18:0, PC ae C36:4, Kynurenine, PC ae C40:2, PC ae C42:5, PC ae, C36:5, lysoPC a C20:3, Cit, lysoPC a C16:0, SM C24:0, C16, PC ae C42:3, PC aa C32:1, Ile, C4, or Creatinine in a sample from the subject.

Also provided is a method of measuring a panel of biomarkers in a female subject at risk of or diagnosed with a neurodegenerative disease, comprising: measuring the level of one or more (e.g., 1, 2, 3, 4, 5, 6, 7, 8, 9, 10, or all) metabolites selected from, for example, PC ae C40:2, SM C16:0, C14:1, alpha-AAA, C4, Trp, Ile, SM C26:0, SM C16:1, PC aa C36:5, or Thr in a sample from the subject.

Other embodiments provide a method of measuring a panel of biomarkers in a APOE e4⁺ subject at risk of or diagnosed with a neurodegenerative disease, comprising: measuring the level of one or more (e.g., 1, 2, 3, 4, 5, 6, 7, 8, 9, 10, 11, 12, 13, 14, 15, 16, 17, 18, or all) metabolites selected from, for example, PC ae C36:0, PC ae C42:5, PC aa C32:0, PC aa C42:6, PC ae C36:5, lysoPC a C16:0, PC aa C36:0, PC aa C32:3, PC ae C34:2, C16:1, lysoPC a C18:0, SM C18:1, PC aa C36:1, PC aa C34:4, PC ae, C36:4, C18:1, PC aa C42:5, SM (OH) C24:1, or PC aa C38:0 in a sample from the subject.

Further embodiments provide a method of measuring a panel of biomarkers in a APOE e4⁻ subject at risk of or diagnosed with a neurodegenerative disease, comprising: measuring the level of one or more (e.g., 1, 2, 3, 4, 5, 6, 7, 8, 9, 10, 11, 12, 13, 14, 15, 16, 17, 18, 19, 20, 21, 22, 23, 24, or all) metabolites selected from, for example, C12, PC aa C36:5, Thr, SM C16:1, SM (OH) C24:1, PC ae C42:4, Ala, PC ae C36:5, Creatinine, PC ae C36:0, Cit, SM C20:2, PC aa C42:0, PC ae C44:3, lysoPC a C16:1, PC aa C40:6, C14:1, C4, PC aa C38:6, lysoPC a C18:1, PC aa C32:1, PC aa C38:0, PC aa C36:6, PC ae C42:2 or PC ae C40:6 in a sample from the subject.

Additional embodiments provide a method of measuring a panel of biomarkers in a male APOE e4⁺ subject at risk of or diagnosed with a neurodegenerative disease, comprising: measuring the level of one or more (e.g., 1, 2, 3, 4, 5, 6, 7, 8, or all) metabolites selected from, for example Taurine, PC ae C36:5, PC aa C42:6, PC aa C38:4, SM (OH) C22:1, PC ae C32:1, PC ae C32:2, lysoPC a C18:0, or Asn in a sample from the subject.

Yet other embodiments provide a method of measuring a panel of biomarkers in a female APOE e4⁺ subject at risk of or diagnosed with a neurodegenerative disease, comprising: measuring the level of one or more (e.g., 1, 2, 3, 4, or all) metabolites selected from, for example, PC aa C34:4, PC ae C40:2, lysoPC a C18:1, PC aa C38:3, or PC aa C42:5 in a sample from the subject.

In certain embodiments, provided herein is a method of measuring a panel of biomarkers in a male APOE e4⁻ subject at risk of or diagnosed with a neurodegenerative disease, comprising: measuring the level of one or more (e.g., 1, 2, 3, 4, 5, 6, 7, 8, 9, 10, or all) metabolites selected from, for example, C14:1-OH, Ala, PC ae C40:4, PC ae C36:0, SM (OH) C24:1, PC ae C40:2, PC ae C38:5, Tyr, Sarcosine, lysoPC a C16:0, or C9 in a sample from the subject.

In further embodiments, provided herein is a method of measuring a panel of biomarkers in a female APOE e4⁻ subject at risk of or diagnosed with a neurodegenerative disease, comprising: measuring the level of one or more (e.g., 1, 2, 3, 4, 5, 6, 7, 8, 9, 10, 11, 12, 13, 14, 15, 16, or all) metabolites selected from, for example, C16:1, SM C16:0, C12, C9, PC ae C36:4, Lys, PC ae C32:2, PC ae C44:6, PC aa C36:5, SM (OH) C14:1, PC aa C34:3, Creatinine, PC ae C36:0, PC aa C28:1, SM C26:0, PC ae C40:3, or SM (OH) C22:2 in a sample from the subject.

In some embodiments, a computer-based analysis program is used to translate the raw data generated by the detection assay (e.g., the presence, absence, or amount of a specific metabolite or group of metabolites) into data of predictive value for a clinician. The clinician can access the predictive data using any suitable means. Thus, in some embodiments, the present invention provides the further benefit that the clinician, who is not likely to be trained in metabolite analysis, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a blood sample) is obtained from a subject and submitted to a profiling service (e.g., clinical lab at a medical facility, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (e.g., metabolic profile), specific for the diagnostic or prognostic information desired for the subject.

The profile data is then prepared in a format suitable for interpretation by a treating clinician. For example, rather than providing raw data, the prepared format may represent a diagnosis or risk assessment (e.g., likelihood of AD or prognosis of AD) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may choose further intervention or counseling based on the results. In some embodiments, the data is used for research use. For example, the data may be used to further optimize the inclusion or elimination of markers as useful indicators of a particular condition or stage of disease.

Embodiments of the present disclosure provide screening applications (e.g., to screen candidate drugs for neurodegenerative diseases). For example, in some embodiments, individuals are administered test compounds or interventions (e.g., dietary interventions). The levels of the metabolites described herein are measured before and after administration of the drug or other intervention to identify interventions that alter levels of the metabolites.

Other embodiments provide for monitoring a subject over time for levels of metabolites (e.g., to determine if a subject has developed a neurodegenerative disease or to monitor the progression of a neurodegenerative disease).

Compositions for use (e.g., sufficient for, necessary for, or useful for) in the diagnostic, research or screening methods of some embodiments of the present invention include reagents necessary, sufficient or useful for detecting the presence or absence of specific metabolites. Any of these compositions, alone or in combination with other compositions of the present invention, may be provided in the form of a kit. Kits may further comprise appropriate controls and/or detection reagents.

Further embodiments provide compositions and methods for treating or preventing neurodegenerative disease (e.g., cognitive decline) in a subject (e.g., a subject identified using the markers described herein). In some embodiments, subjects are treated with a composition comprising one or more of the patient specific agents described in Tables 10-15. In some embodiments, the composition is formulated as a pharmaceutical composition. In some embodiments, the composition is formulated as a food, food product, or nutraceutical.

In some embodiments, provided herein are pharmaceutical compositions comprising of one or more compounds described herein and a pharmaceutically acceptable carrier. In some embodiments, compositions are formulated for administration by any suitable route, including but not limited to, orally (e.g., such as in the form of tablets, capsules, granules or powders), sublingually, bucally, parenterally (such as by subcutaneous, intravenous, intramuscular, intradermal, or intracisternal injection or infusion (e.g., as sterile injectable aqueous or non-aqueous solutions or suspensions, etc.)), nasally (including administration to the nasal membranes, such as by inhalation spray), topically (such as in the form of a cream or ointment), transdermally (such as by transdermal patch), rectally (such as in the form of suppositories), etc.

The present disclosure is not limited to a particular formulation comprising one or more of the above-described compositions. In some embodiments, compositions are provided as one or more of supplements, food products, foods, and food additives. In some embodiments, foods and food products are one or more of bars (e.g., raw bars), biscuits, crackers, chips, pastes, gruels and liquids beverages, powders, and the like.

In some embodiments, compositions are provided as powders or pastes that can be mixed with a liquid to provide a beverage.

The dietary supplement may comprise one or more inert ingredients, especially if it is desirable to limit the number of calories added to the diet by the dietary supplement. For example, the dietary supplement may also contain optional ingredients including, for example, herbs, vitamins, minerals, enhancers, colorants, sweeteners, flavorants, inert ingredients, and the like.

### EXPERIMENTAL

The following examples are provided in order to demonstrate and further illustrate certain preferred embodiments and aspects of the present disclosure and are not to be construed as limiting the scope thereof.

### Methods

### Differential Expression Analysis

The metabolite abundance after normalization and covariate adjustment are subjected to t-test using Limma R package. After adjustment, AD and CN samples were extracted in each group. The t-test is performed on all metabolites in each group between AD and CN samples.

### Empirical/Non-Parametric Bootstrap Method

For patient group, 100 networks, which were based on randomly sampling 90 percent of the samples, were built. To evaluate the confidence interval of each edge, an empirical bootstrap method was performed by bootstrapping the vector of posteriors of each edge across 100 networks for 1000 times. The bootstrap procedure results in a good approximation of the background distribution of the edge posterior, from which the interval of the edge posterior with 95% confidence level was derived.

### Biomarker Panel Selection

The permutation importance method in python package eli5 was used to quantify the importance of features by calculating the performance loss with feature value shuffling. The performance deterioration quantified the importance of the feature. Next, the selected features were used to train elastic net model with feature selection in 10-fold cross-validation per patient group. The prediction performance is evaluated by area under curve (AUC).

Biomarker Association with Clinical Features

For each biomarker panel, principal components explained >90% of the variance in data. The response variables (clinical cognition and CSF pathology variables) was regressed on these principal components and ANOVA with F-statistics was used to calculate the fitness of regression.

### Predictive Network Analysis

The predictive network model integrates the conventional Bayesian Network (BN) and bottom-up causality inference algorithm [98, 99] to represents a joint probability distribution over a set of random variables by a causal graph structure and a vector of parameters. The conventional Bayesian network has been a popular method to infer biological network from various multivariate omics data. However, one major bottleneck of BN is the indistinguishable causality among alternative structures when they become statistically equivalent (equivalent class), which resulted in only partially causal structures.

To tackle this problem, a bottom-up belief propagation engine as a subroutine to infer causality [100, 101 US2019019864] among these alternative structures was used. The bottom-up method leverages the nonlinearity of biochemical reactions to infer causality of a molecular interaction, fitting better to the data along true causal direction than false causal direction, which breaks the statistical equivalence in Bayesian network. By integrating the novel bottom-up causality inference approach with top-down BN, the integrated top-down and bottom-up predictive network pipeline result in complete/full causal network with discerned causality among the equivalent structures otherwise indiscernible in BN. This pipeline inherits the advantage of BN to integrate the multi-scale ‘omics-’ data (genetics, genomics, proteomics, metabolomics, epigenomics) to construct multi-scale network models. Consequently, the nodes in a predictive network can represent any variable of interest within the biological system, e.g. levels of gene expression, the genotype of a locus, the activity of a protein, or the abundance of a metabolite.

### Example 1

### ADNI Cohort and Blood-Based Metabolite Measurements

The ADNI study (adni.loni.usc.edu/) aims to identify de-novo biomarkers of mild cognitive impairment (MCI) and early Alzheimer's disease (AD) by collecting longitudinal data of magnetic resonance imaging (MRI), positron emission tomography (PET), blood-based metabolite and clinical assessment along with the progression of cognitive decline.

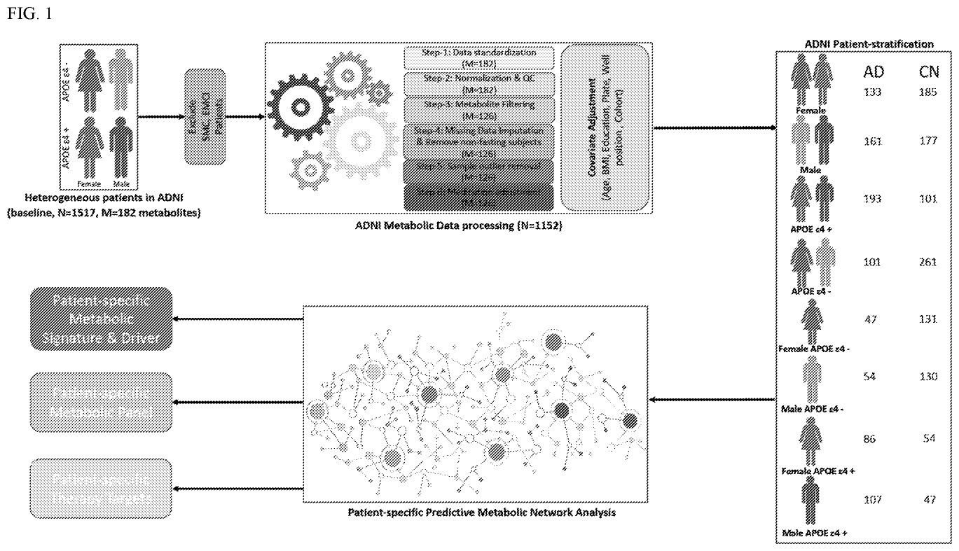

In this study, the blood-based metabolic data generated by Biocrate P180 assay from 1517 baseline samples of fasting subjects in ADNI phase 1, and 2/GO was normalized by utilizing the computational pipeline described in (Toledo, J. B., et al., Metabolic network failures in Alzheimer's disease: A biochemical road map. Alzheimers Dement, 2017. 13 (9): p. 965-984) and focusing analyses on baseline cognitive normal (CN) and AD samples (total 656 samples including 362 CN and 294 AD). The normalized data are further processed by removing the co-founding factors of age, body mass index (BMI), education, plate and well position in each phase (FIG. 1). The residuals of each phase are combined, and effect of phase (ADNI-1 and ADNI-2/GO) is removed from the combined residual to derive the final ADNI residual.

### AD Patient Stratification by Sex and APOE Genotype

To investigate the sex and APOE genotype-specific metabolic association with AD, 656 samples were stratofed into 8 groups defined by sex and APOE genotype in two hierarchical layers (summarized in Table 1): i) in the first layer, the samples were stratified according to sex or APOE genotype respectively: males only, females only, APOE ε4 carriers and APOE & 4 non-carriers; ii) in the second layer, samples were further stratified according to the combination of sex and APOE genotype: male APOE ε4 carriers, male APOE ε4− non-carriers, female APOE ε4 carriers and female APOE ε4− non-carrier. In addition, the background metabolic association with AD in all 656 samples was analyzed without patient stratification. Comparison of patient-specific analysis to the background signal further illustrated the multi-modality of the metabolic association with AD affected by sex and APOE 84 genotype.

### Sex and APOE Genotype-Specific Metabolic Signature of AD

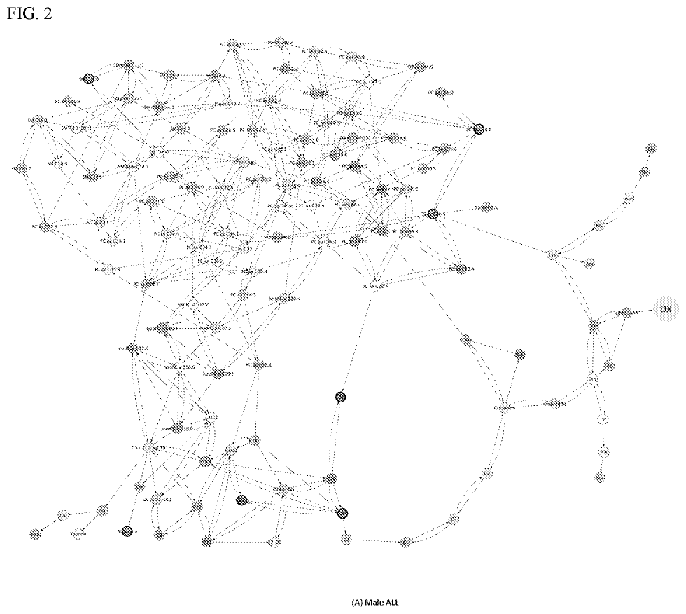

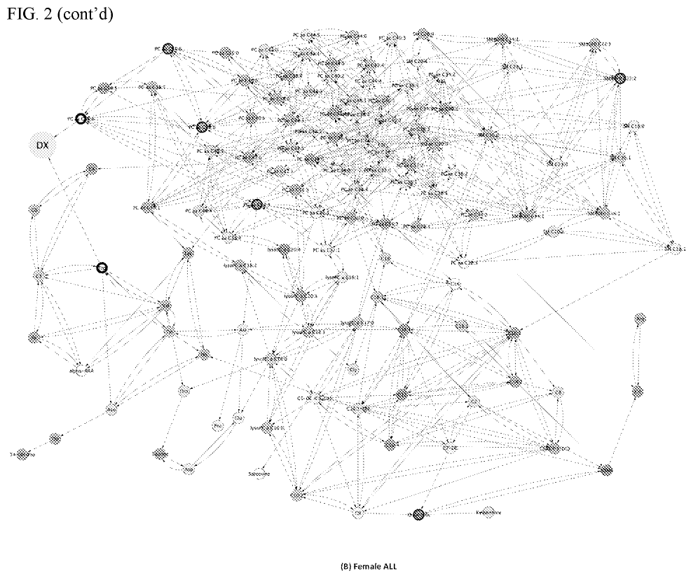

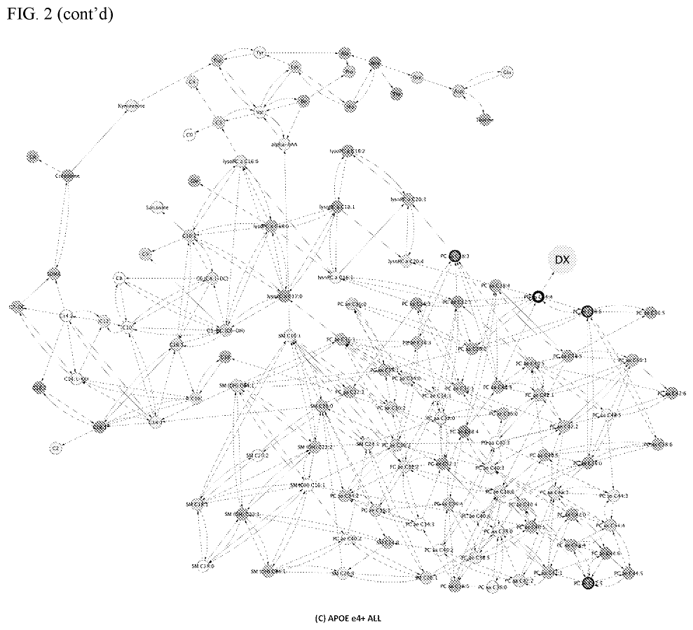

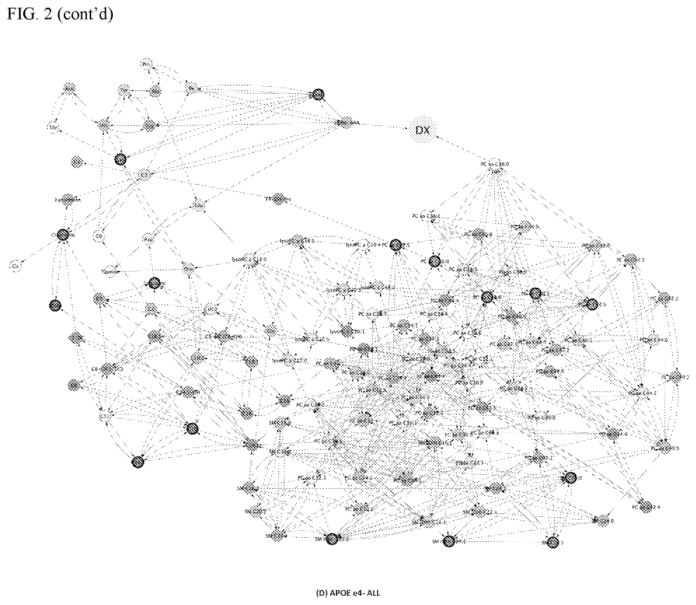

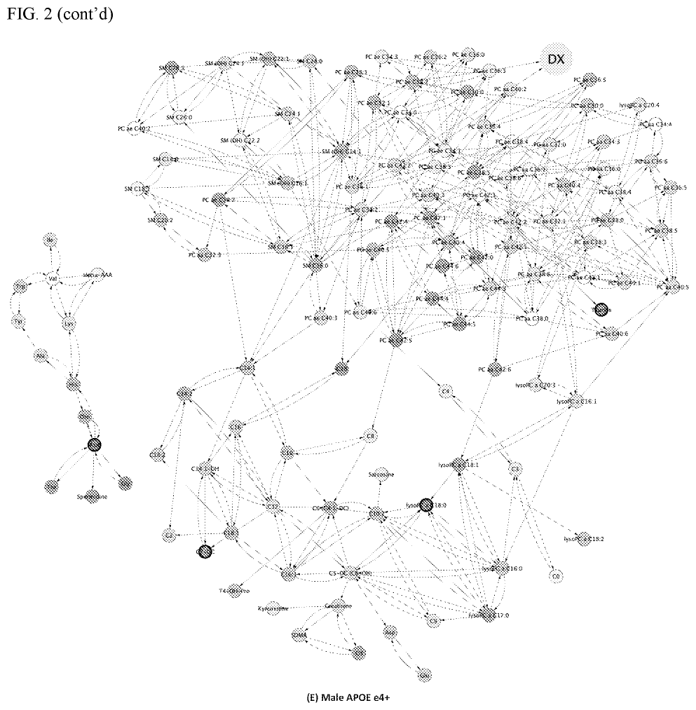

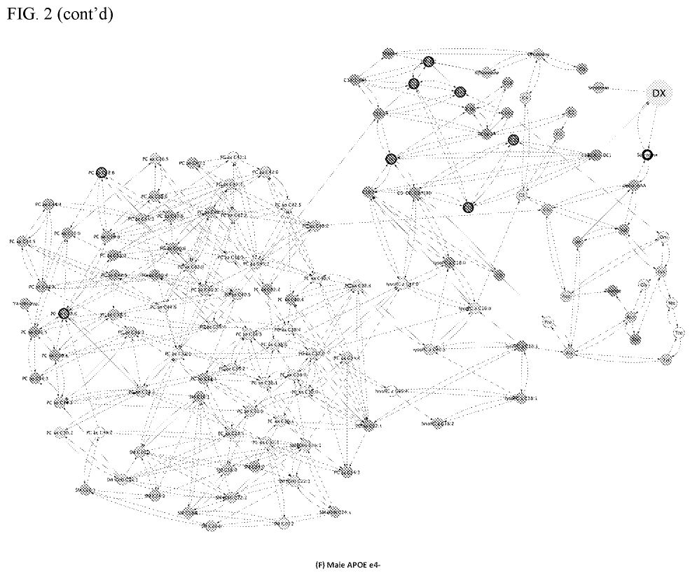

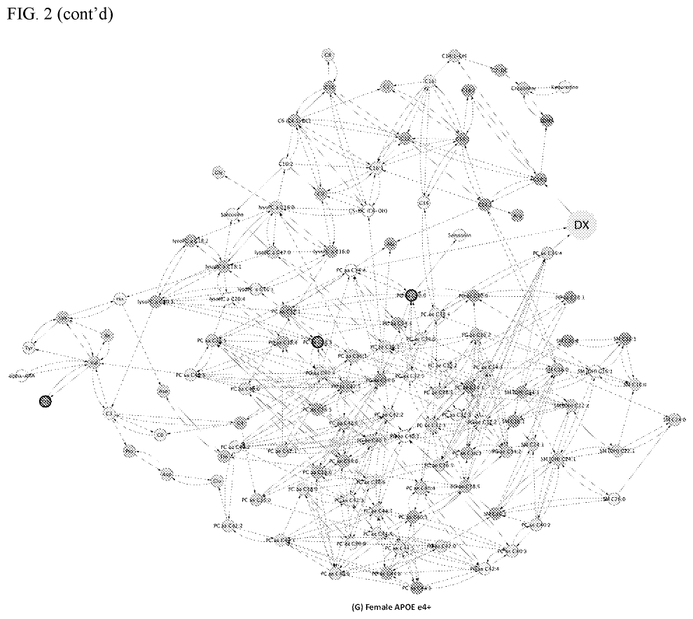

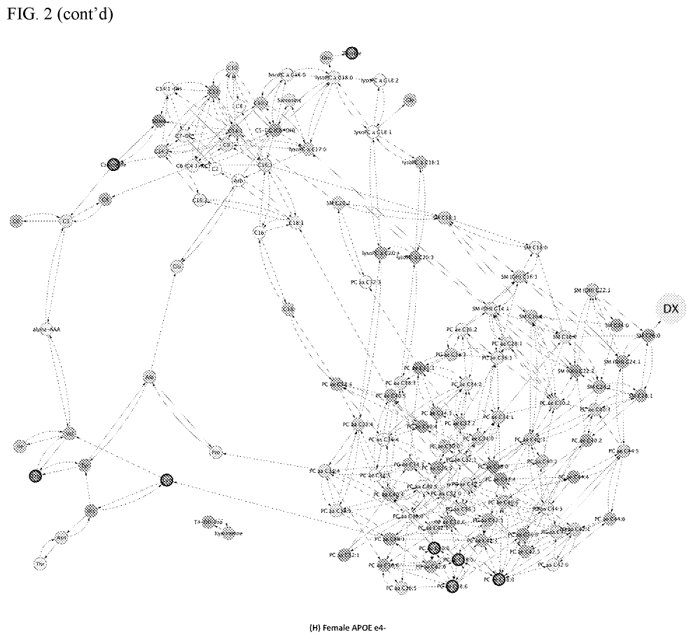

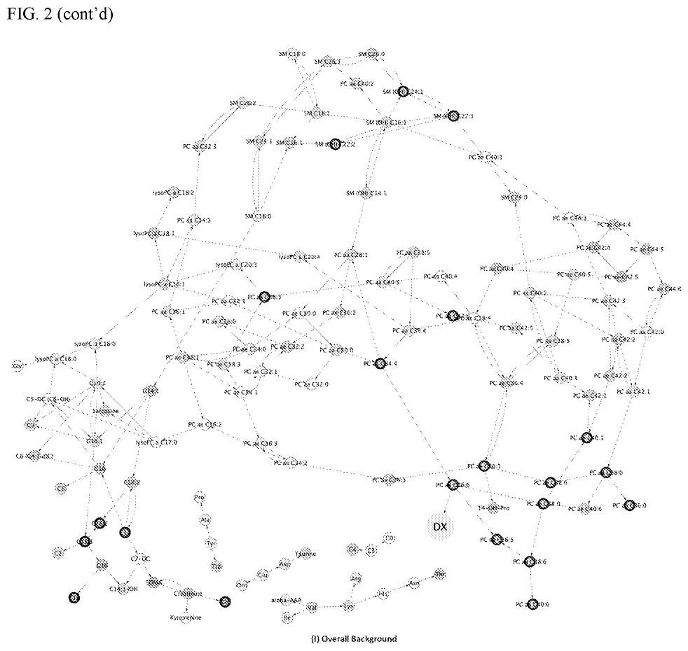

Metabolic changes in the blood of AD patients have been demonstrated to reflect the structural and functional degeneration in the brain (Arnold, M., et al., Sex and APOE epsilon4 genotype modify the Alzheimer's disease serum metabolome. Nat Commun, 2020. 11 (1): p. 1148). However, the human blood-based metabolic signature associated with AD has not been established, which is partially due to the convolution of metabolic changes in the blood with complex molecular mechanism underlying the onset and progression of AD that are affected by multiple factors, such as sex and genotype. To untangle sex and APOE genotype difference in metabolic changes, differential expression (DE) analysis was performed on the metabolic residuals in ADNI after normalization and covariate adjustment. The residuals of AD to CN samples was compared to identify metabolic DE signature in each of the 8 groups described above (FIG. 2).

### Overall Background Metabolic Signature of AD

By analyzing all 656 samples, a unified metabolic signature of AD across all sexes and APOE ε4 genotypes, which primarily consist of lipids including significant down-regulation of 13 PCs (PC aa C36:6, PC ae C38:0, PC ae C36:5, PC aa C38:6, PC ae C38:6, PC aa C40:6, PC aa C38:0, PC aa C42:6, PC aa C36:5, PC aa C36:0, PC aa C38:3, PC ae C40:1, PC aa C34:4), significant down-regulation of three SMs (SM (OH) C22:2, SM (OH) C24:1, SM (OH) C22:1), significant up-regulation of four acyl-carnitines (C18, C18:2, C12, C18:1) and significant up-regulation of citrulline was identified.

### Sex-Specific Metabolic Signature of AD

It was found that metabolic signatures of AD differed in Male and Female patients. In males, three acyl-carnitines (C18, C18:1 and C18:2) were significantly (p-value<0.05) up-regulated in AD compared to CN. Sarcosine, a derivative of amino acid primary found in muscle tissue, was significantly down-regulated in AD males, and branched-chain amino acid (BCAA) valine was down-regulated for more than 7-fold in AD though not statistically significant indicating it may play a role in amino acid metabolism and energy production in male AD patients. In addition to amino acids, two PCs (PC ae C36:5 and PC ae C38:6) and one SM (SM C24:0) was significantly down-regulated in AD males. In females, a prominent lipid signature consisting of four significantly down-regulated PCs (PC aa C36:6, PC ae C38:0, PC aa C38:6, PC aa C34:4) and one significantly down-regulated SM (SM (OH) C22:2) was identified. In addition to lipids, L-tryptophan an essential amino acid, was significantly down-regulated in AD females compared to CN females. Creatinine produced by breakdown of creatine in muscles, was significantly up-regulated in AD females. Stratification based on sex demonstrated that males and females AD patient metabolize distinctive class of metabolites for mitochondrial energy production.

### APOE-Specific Metabolic Signature of AD

In APOE ε4 non-carrier group (APOE ε4−), a heterogenous classes of metabolites that changed their levels in the blood, including significant down-regulation of nine phosphorytidycholines (PC aa C38:0, PC aa C38:6, PC ae C 38:6, PC aa C40:6, PC aa C36:0, PC ae C38:0, PC ae C36:5, PC ae C40:1, PC aa C36:6), four sphingomylines (SM (OH) C24:1, SM C26:1, SM (OH) C22:2, SM C26:0), and three amino acids (sarcosine, lysine, valine), and significant up-regulation of creatinine and L-citruline, three acyl-carnitines (C10, C12, and C14:2), and a proinflammatory agent symmetric dimethylarginine (SDMA), which has been linked to type-2 diabetics, chronical kidney disease and cardiovascular disease [66, 67] was identified. In APOE 24 carriers (APOE ε4+), a homogeneous lipid profile consisting of significantly down-regulated phosphorytidycholines (PC aa C34:4, PC aa C38:3 and PC aa C36:6) and significantly up-regulated PC ae C42:5 was identified.

### Sex- and APOE-Specific Metabolic Signature of AD

In male APOE ε4 carriers, significantly down-regulated taurine and significantly up-regulated asparagine was observed. Besides amino acids, the metabolic signature also features significantly up-regulated lyso-PC a C18:0 and significantly down-regulated acyl-carnitine C7-DC. In male APOE ε4 non-carriers, significant up-regulation of 6 acyl-carnitines (C7-DC, C8, C10, C12, C14:2, C16:1), and significant down-regulation of two PCs (PC aa C38:6 and PC ae C38:6) and significant down-regulation of sarcosine was identified. The branched-chain amino acid (valine) was down-regulated more than 13-fold with close-to-be statistical significance (p-value=0.058). The amino acid taurine is up-regulated by more than 9-fold and close to being statistically significant (p-value-0.062).

In female APOE ε4 carriers, a consistent lipid signature of significant down-regulation of three PCs (PC aa C34:4, PC aa C30:0, PC aa C38:3) and the essential amino acid L-tryptophan was identified. In female APOE ε4 non-carriers, significant alteration in amino acids including down-regulation of L-tryptophan, lysine and taurine, and significant up-regulation of L-citrulline and creatinine was identified. Besides significantly altered amino acids, this signature also includes significant down-regulation of four PCs (PC aa C38:0, PC aa C40:6, PC ae C38:0, PC aa C38:6).

### Consensus Sex and APOE-Specific Metabolic Network in AD

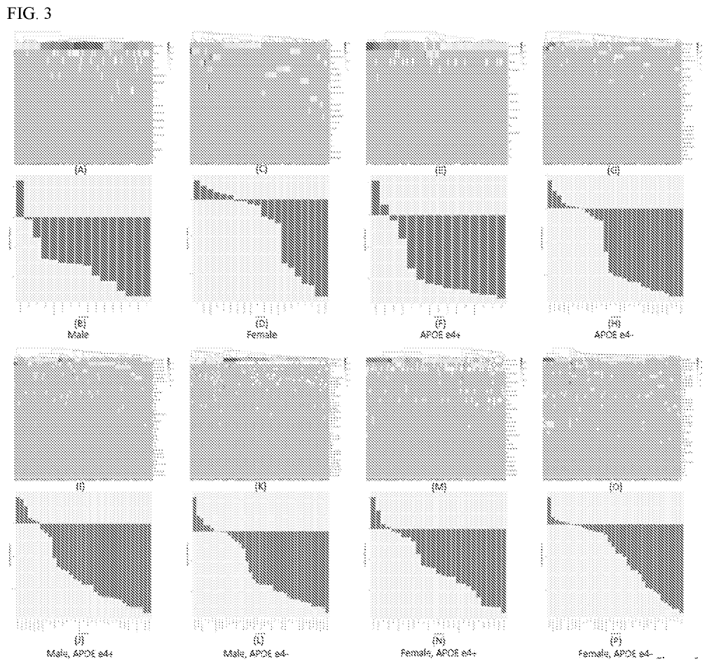

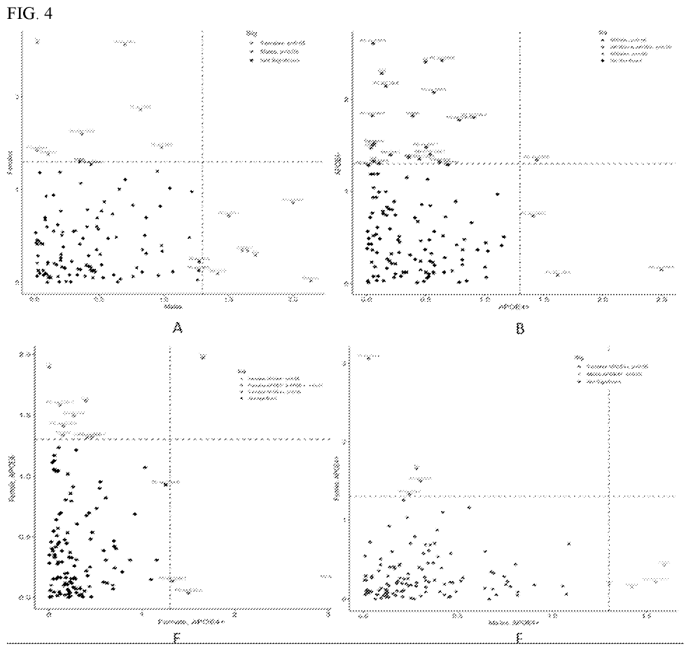

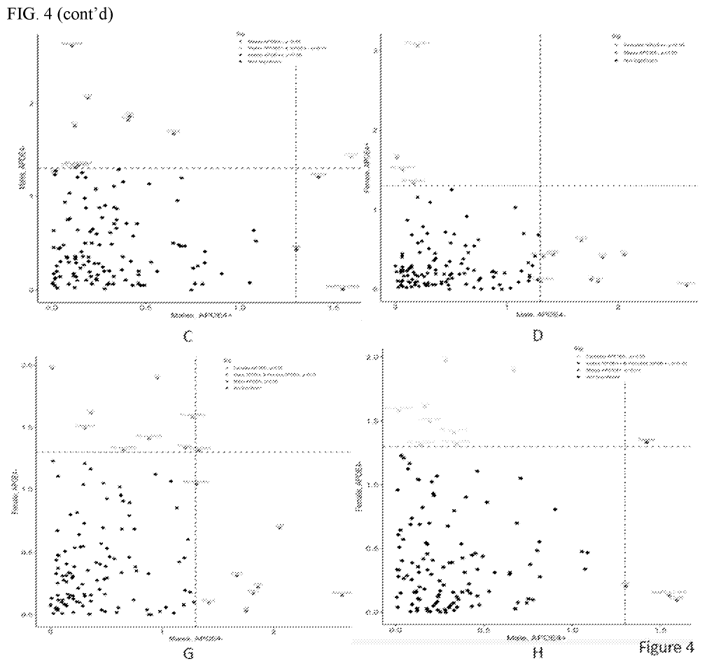

While DE analysis revealed metabolic signature associated with AD in each group, the power of differential expression analysis to detect a small-to-moderate metabolic variance in the ADNI data is small. In addition, metabolic signatures cannot distinguish whether a metabolite is an upstream driver or subsequent effector associated with AD pathology. The upstream driver metabolites of diagnosis (Dx) node in the network indicate potential drivers to modulate AD pathology or biomarkers of AD state. Therefore, distinguishing driver metabolites could give insights leading to development of a precision biomarker panel and therapeutic targets in each group. To this end, casual metabolic network models were built using an published predictive network modeling pipeline [2, 4, 63, 64] by integrating the metabolic residuals of 362 CN and 294 AD samples. To investigate the robustness of network models and metabolic drivers, 100 sub-samplings were performed to generate simulated population-based distributions for metabolic interactions. In each sub-sampling, 90% of the 362 CN and 294 AD samples in each patient group were randomly sampled to construct predictive network model. In total, 100 causal network models in each patient group were developed. By pooling edges from these 100 networks, the background distribution of metabolite interactions (edges) was formed and the 95% confidence interval of every network edge per patient group with non-parametric 1000 bootstrapping was calculated. To derive the patient-specific consensus metabolic networks, 80% of edges with 95% of confidence level per patient group were included. In addition, the background metabolic network was built by using all 656 samples (FIG. 3, Supplementary Table S3). By comparing this background network to the patient-specific network, a panel of sex- and APOE ε4-specific upstream metabolites associated with AD in each patient group was identified (FIG. 4). The background metabolic network identified a subnetwork of PCs mediated by PC aa C36:6 was associated with AD.

Metabolic Network and Driver Associated with AD in Male and Female

In males, the consensus metabolic network identified a subnetwork of down-regulated branched-chain amino acids (BCAAs), including alpha-AAA, Valine, Isoleucine and Lysine, was associated with AD. In females, the consensus network model identified a subnetwork of PCs mediated by PC aa C36:6 and downregulation of L-tryptophan was associated with AD.

Metabolic Network and Driver Associated with AD in APOE e4 Carrier and Non-Carrier

In APOE ε4 carriers, the consensus network model identified a subnetwork of PCs mediated by PC aa C34:4 was associated with AD, where in APOE ε4 non-carriers, the consensus network model identified a subnetwork of PCs mediated by PC aa C38:0 was associated with AD.

Metabolic network and driver associated with AD in Male/Female APOE e4 carrier/non-carrier Further stratification of AD and CN patients by sex and APOE genotype demonstrated that in male APOE 84 carriers, the consensus metabolic network identified a subnetwork of PCs mediated by PC ae C36:3, PC aa C40:2, PC ae C42:5 (16%), was associated with AD. In male APOE ε4 non-carriers, the consensus metabolic network identified a subnetwork of acyl-carnitines mediated by C6 (C4:1-DC) and a subnetwork of BCAAs and biogenic amine mediated by sarcosine, was associated with AD. Note that, alpha-AAA is direct upstream of sarcosine in this network. In particularly, valine, which is direct upstream of alpha-AAA is down-regulated over 13-fold in AD male non-carriers with close-to-be significant (p-value=0.058).

In female APOE ε4 carriers, the consensus network identified a subnetwork of PCs mediated by PC ae C36:4 and PC aa C34:4, and a subnetwork of acyl-carnitines mediated by C14:1, as well interestingly, the significant downregulation (FC=−5.34, p-value=0.0227) of L-tryptophan and thusly decline of de-novo synthesis of NADH/NAD+ via kynurenine metabolism pathway (Weaver, D., et al., Alzheimer's Disease as a Disorder of Tryptophan Metabolism (2745). Neurology, 2020. 94 (15 Supplement): p. 2745; Porter, R. J., et al., Cognitive deficit induced by acute tryptophan depletion in patients with Alzheimer's disease. Am J Psychiatry, 2000. 157 (4): p. 638-40; Okamoto, H., F. Okada, and O. Hayaishi, Kynurenine metabolism in hyperthyroidism. A biochemical basis for the low NAD (P) level in hyperthyroid rat liver. J Biol Chem, 1971. 246 (24): p. 7759-63) was associated with AD, which is consistent to the female as a whole. In female APOE ε4 non-carriers, the consensus network discovered a subnetwork of SMs and PCs mediated by SM C26:0 and SM C16:1, was associated with AD.

Sex and APOE-Specific Blood-Based Metabolic Biomarker Panel Associated with Clinical Cognitive Assessment

To establish patient-specific blood-based metabolic biomarkers, a pipeline to identify patient-specific metabolite biomarkers whose measurements in the serum predicted AD diagnosis at baseline in each patient group was developed by training machine learning model to select a subset of features by integrating metabolic signatures, metabolic network structures and clinical features (age, body-mass-index (BMI), and length of education) and compare their prediction accuracy: i) all 127 metabolites in the data; ii) differential metabolic signature; iii) network structure-derived drivers and effectors; iv) combination of ii) and iii); v) combination of iii) and clinical features; vi) combination of ii), iii) and clinical features. To evaluate the prediction accuracy of every patient-specific panel, 1000 times subsampling was performed to split the total samples into 80% training data and 20% validation data in each patient group and the prediction performance was evaluated by calculating the true and false positive rate and the averaged AUC over 1000 subsampling (FIG. 5). It was found that i) network structure-derived features achieved better prediction accuracy than all features in 5 out of 8 patient groups (male all, APOE e4+ all, APOE e4− all, female APOE e4+ and female APOE e4−), and similar performance for female all; Only in male APOE e4+ and male APOE e4− groups, all features achieved slightly better prediction than network structure-derived features; ii) The best prediction accuracy is achieved by combination of network structure-derived features with clinical features (age, BMI and length of education) in 7 out of 8 patient groups except male APOE e4− where the prediction accuracy is only slightly lower than the best performance achieved by all features; Note that in female APOE e4− group, combination of network-derived with clinical features improved the prediction accuracy by more than 0.6 in AUC comparing to those from all features; iii) significant metabolic signature alone generally produced the worst prediction accuracy in all patient groups; iv) combination of metabolic signature with other features generally degraded the prediction accuracy than those without signature across patient groups. In summary, these data indicate that the metabolic network-derived features with or without clinical features (age, BMI, education length) generally outperforms other feature sets and can be used as patient-specific biomarker panels (Tables 3-10).

Blood-Based Metabolic Biomarker Panel Associated with Cognition Decline

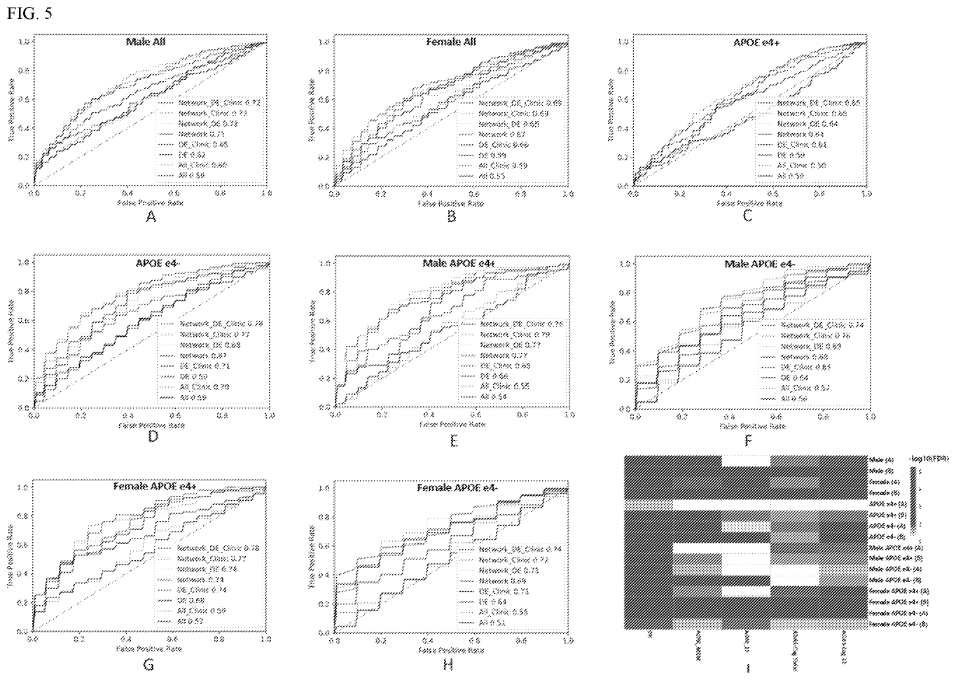

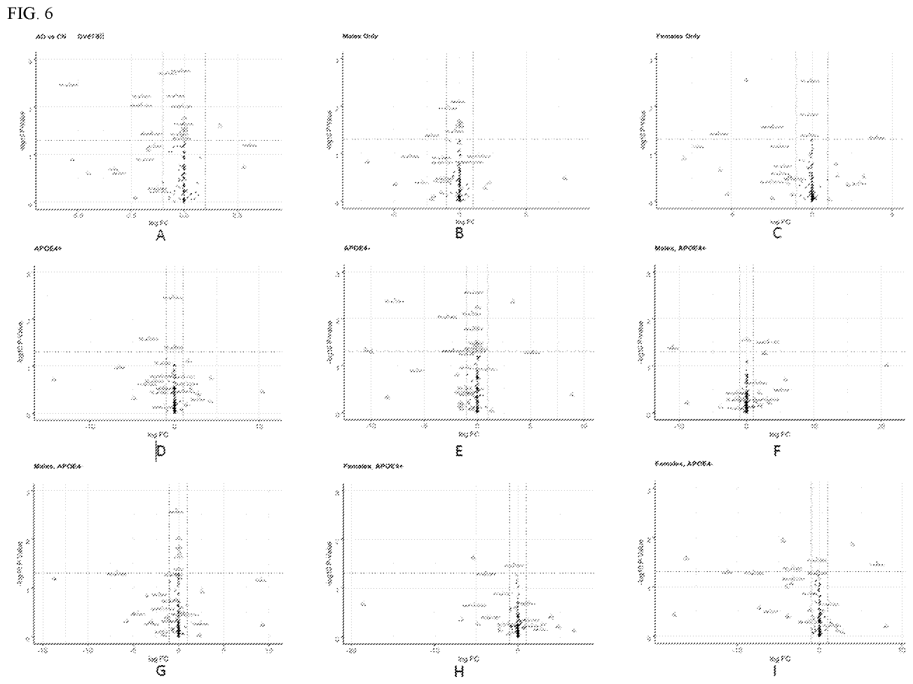

To investigate the association of the biomarker panels with the AD pathology (CSF Aβ 1-42 and CSF p181-Tau) and with cognition (Total Score, Total13, ADNI_MEM, ADNI_EF) in ADNI cohort, the eigen metabolic expression level, which captured the primary variance component for every panel and fitted a linear regression model between eigen expression level and pathology and cognition scores. The significance of association is summarized in FIG. 6: 1) the biomarker panels of each patient group derived from network model with or without additional clinical measurements (Age, BMI, and Education) were all significantly associated with the diagnosis (Dx); 2) Out of the total 16 biomarker panels over 8 patient groups (2 panels per group), 15 panels were significantly associated with the memory (ADNI_MEM) except network-derived panel in Female APOE e4 carriers (p-value=0.085); 3) Out of the 16 biomarker panels, 15 panels were significantly associated with the overall cognition test score (Total Score) except male APOE e4 non-carriers (p-value=0.068), whereas all 16 panels were significantly associated with the 13-cognition test score (Total 13); 4) Out of 16 panels, 10 panels were significantly associated with the executive function (ADNI_EF). Among the rest 6 panels, it is noted that three panels are associated with ADNI_EF with close to being statistically significant, i.e. network-derived biomarkers of Male APOE e4 carriers (p-value=0.0557), of male APOE e4 non-carriers (p-value=0.0587) and of female APOE e4 non-carriers (p-value=0.063); 4) The network-derived biomarkers with clinical features of male APOE e4 non-carriers are significantly associated with CSF P181-Tau level (p-value=0.014) and network-derived biomarkers of females are very close to be statistically significantly associated with CSF P181-Tau level (p-value=0.052), although CSF Aβ and pTau level changes precede clinical manifestation, indicating our signature could be associated with AD pathology for certain patient group.

Overall, the results indicate that measuring levels of patient-specific metabolite biomarkers in the serum can accurately predict AD diagnosis and were associated with memory and executive functions. In some patient groups, they are also associated with CSF changed related to AD pathology. Therefore, the biomarker panels are useful in diagnosis of AD and in the evaluation of cognitive declines.

TABLE 1
Characteristics of the 1152 ADNI subjects in this study

| Demographics | CN | | LMCI | | AD | |
|---|---|---|---|---|---|---|
| Sample size | 362 | | 496 | | 294 | |
| Sex (m/f) | 177/185 | | 307/189 | | 161/133 | |
| Age | 74.61 | (+/−5.66) | 74.11 | (+/−7.57) | 74.71 | (+/−7.85) |
| BMI | 27.04 | (+/−4.51) | 26.49 | (+/−4.32) | 26.87 | (+/−4.71) |
| Education | 16.20 | (+/−2.79) | 15.86 | (+/−2.91) | 15.19 | (+/−2.99) |
| APOE ε4 +/− | 101/261 | | 265/231 | | 193/101 | |
| Clinic Cognition | | | | | | |
| ADAS-Cog | 5.99 | (+/−3.04) | 11.56 | (+/−4.50) | 19.34 | (+/−6.75) |
| total score | | | | | | |
| ADAS-Cog | 9.25 | (+/−4.37) | 18.72 | (+/−6.51) | 29.68 | (+/−8.11) |
| 13 score | | | | | | |
| Memory function | 0.95 | (+/−0.53) | −0.05 | (+/−0.57) | −0.73 | (+/−0.52) |
| (ADNI_MEM) | | | | | | |
| Executive | 0.74 | (+/−0.70) | 0.014 | (+/−0.78) | −0.82 | (+/−0.84) |
| function | | | | | | |
| (ADNI_EF) | | | | | | |
| CSF Pathology | | | | | | |
| CSF p181-Tau | 25.54 | (+/−14.80) | 35.36 | (+/−17.36) | 41.64 | (+/−19.63) |
| CSF Aβ1-42 | 207.67 | (+/−54.47) | 163.48 | (+/−53.57) | 143.64 | (+/−41.86) |

TABLE 2

| Group | | | APOE | AD | CN |
|---|---|---|---|---|---|
| Index | Patient Group | Sex | e4 | Sample | Sample |
| 1 | Male All | Male | 0/1/2 | 161 | 177 |
| 2 | Fernale All | Female | 0/1/2 | 133 | 185 |
| 3 | Non-Carrier All | Male/Female | 0 | 101 | 261 |
| 4 | Carrier All | Male/Female | 1/2 | 193 | 101 |
| 5 | Male Carrier | Male | 1/2 | 107 | 47 |
| 6 | Male Non-carrier | Male | 0 | 54 | 130 |
| 7 | Female Carrier | Female | 1/2 | 86 | 54 |
| 8 | Female Non-carrier | Female | 0 | 47 | 131 |

TABLE 3
Male

| Metabolite Only | Full_Name |
|---|---|
| C18 | Octadecanoylcarnitine |
| PC aa C42:6 | Phosphatidylcholine aa C42:6 |
| lysoPC a C18:0 | Lysophosphatidylcholine a C18:0 |
| PC ae C36:4 | Phosphatidylcholine ae C36:4 |
| Kynurenine | Kynurenine |
| PC ae C40:2 | Phosphatidylcholine ae C40:2 |
| PC ae C42:5 | Phosphatidylcholine ae C42:5 |
| PC ae C36:5 | Phosphatidylcholine ae C36:5 |
| lysoPC a C20:3 | Lysophosphatidylcholine a C20:3 |
| Cit | Citrulline |
| lysoPC a C16:0 | Lysophosphatidylcholine a C16:0 |
| SM C24:0 | Sphingomyelin C24:0 |
| C16 | Hexadecanoylcarnitine |
| PC ae C42:3 | Phosphatidylcholine ae C42:3 |
| PC aa C32:1 | Phosphatidylcholine aa C32:1 |
| Ile | Isoleucine |
| C4 | Butyrylcarnitine |
| Creatinine | Creatinine |
| Metabolite & Clinical | Full_Name |
| PTEDUCAT | PTEDUCAT |
| lysoPC a C18:0 | Lysophosphatidylcholine a C18:0 |
| C18 | Octadecanoylcarnitine |
| PC ae C42:3 | Phosphatidylcholine ae C42:3 |
| PC ae C42:5 | Phosphatidylcholine ae C42:5 |
| PC ae C36:5 | Phosphatidylcholine ae C36:5 |
| Val | Valine |
| PC aa C42:6 | Phosphatidylcholine aa C42:6 |
| PC ae C40:2 | Phosphatidylcholine ae C40:2 |
| SM C24:0 | Sphingomyelin C24:0 |
| Creatinine | Creatinine |
| lysoPC a C16:0 | Lysophosphatidylcholine a C16:0 |
| lysoPC a C20:3 | Lysophosphatidylcholine a C20:3 |
| lysoPC a C16:1 | Lysophosphatidylcholine a C16:1 |
| SM(OH) C24:1 | Hydroxysphingomyelin C24:1 |
| lysoPC a C18:1 | Lysophosphatidylcholine a C18:1 |
| lysoPC a C18:2 | Lysophosphatidylcholine a C18:2 |
| PC ae C38:5 | Phosphatidylcholine ae C38:5 |
| Trp | Tryptophan |
| PC aa C32:1 | Phosphatidylcholine aa C32:1 |
| Lys | Lysine |
| C16 | Hexadecanoylcarnitine |
| SM C26:1 | Sphingomyelin C26:1 |

TABLE 4
Female

| Metabolite | Full_Name |
|---|---|
| PC ae C40:2 | Phosphatidylcholine ae C40:2 |
| SM C16:0 | Sphingomyelin C16:0 |
| C14:1 | Tetradecenoylcarnitine |
| alpha-AAA | alpha-Aminoadipic acid |
| C4 | Butyrylcarnitine |
| Trp | Tryptophan |
| Ile | Isoleucine |
| SM C26:0 | Sphingomyelin C26:0 |
| SM C16:1 | Sphingomyelin C16:1 |
| PC aa C36:5 | Phosphatidylcholine aa C36:5 |
| Thr | Threonine |
| Metabolite + Clinic | Full_Name |
| SM C16:0 | Sphingomyelin C16:0 |
| Thr | Threonine |
| Trp | Tryptophan |
| PC ae C38:0 | Phosphatidylcholine ae C38:0 |
| BMI.bl | Body Mass Index |
| PTEDUCAT | Education Length in Years |

TABLE 5
APOE e4+

| Metabolite | Full_Name |
|---|---|
| PC ae C36:0 | Phosphatidylcholine ae C36:0 |
| PC ae C42:5 | Phosphatidylcholine ae C42:5 |
| PC aa C32:0 | Phosphatidylcholine aa C32:0 |
| PC aa C42:6 | Phosphatidylcholine aa C42:6 |
| PC ae C36:5 | Phosphatidylcholine ae C36:5 |
| lysoPC a C16:0 | Lysophosphatidylcholine a C16:0 |
| PC aa C36:0 | Phosphatidylcholine aa C36:0 |
| PC aa C32:3 | Phosphatidylcholine aa C32:3 |
| PC ae C34:2 | Phosphatidylcholine ae C34:2 |
| C16:1 | Hexadecenoylcarnitine |
| lysoPC a C18:0 | Lysophosphatidylcholine a C18:0 |
| SM C18:1 | Sphingomyelin C18:1 |
| PC aa C36:1 | Phosphatidylcholine aa C36:1 |
| PC aa C34:4 | Phosphatidylcholine aa C34:4 |
| PC ae C36:4 | Phosphatidylcholine ae C36:4 |
| C18:1 | Octadecenoylcarnitine |
| PC aa C42:5 | Phosphatidylcholine aa C42:5 |
| SM(OH) C24:1 | Hydroxysphingomyelin C24:1 |
| PC aa C38:0 | Phosphatidylcholine aa C38:0 |
| Metabolite + Clinic | Full_Name |
| PC ae C42:5 | Phosphatidylcholine ae C42:5 |
| PC aa C42:6 | Phosphatidylcholine aa C42:6 |
| PC aa C32:3 | Phosphatidylcholine aa C32:3 |
| PC aa C32:0 | Phosphatidylcholine aa C32:0 |
| lysoPC a C16:0 | Lysophosphatidylcholine a C16:0 |
| PC aa C42:5 | Phosphatidylcholine aa C42:5 |
| C16:1 | Hexadecenoylcarnitine |
| BMI.bl | Body Mass Index |
| PC ae C34:2 | Phosphatidylcholine ae C34:2 |
| PTEDUCAT | Education length in years |
| PC ae C36:5 | Phosphatidylcholine ae C36:5 |
| PC aa C36:0 | Phosphatidylcholine aa C36:0 |
| PC ae C36:0 | Phosphatidylcholine ae C36:0 |
| PC ae C36:1 | Phosphatidylcholine ae C36:1 |
| PC ae C38:3 | Phosphatidylcholine ae C38:3 |
| SM C18:0 | Sphingomyelin C18:0 |
| SM(OH) C24:1 | Hydroxysphingomyelin C24:1 |
| SM C16:1 | Sphingomyelin C16:1 |
| lysoPC a C17:0 | Lysophosphatidylcholine a C17:0 |
| PC aa C38:4 | Phosphatidylcholine aa C38:4 |
| PC ae C30:2 | Phosphatidylcholine ae C30:2 |
| PC ae C36:4 | Phosphatidylcholine ae C36:4 |
| PC ae C42:1 | Phosphatidylcholine ae C42:1 |
| lysoPC a C18:1 | Lysophosphatidylcholine a C18:1 |
| SM(OH) C14:1 | Hydroxysphingomyelin C14:1 |
| PC aa C38:5 | Phosphatidylcholine aa C38:5 |
| SM(OH) C16:1 | Hydroxysphingomyelin C16:1 |
| PC aa C34:3 | Phosphatidylcholine aa C34:3 |
| SM C16:0 | Sphingomyelin C16:0 |
| C14:1 | Tetradecenoylcarnitine |
| PC ae C40:2 | Phosphatidylcholine ae C40:2 |
| PC aa C40:5 | Phosphatidylcholine aa C40:5 |
| SM(OH) C22:2 | Hydroxysphingomyelin C22:2 |
| PC ae C38:6 | Phosphatidylcholine ae C38:6 |
| PC aa C40:6 | Phosphatidylcholine aa C40:6 |
| PC ae C32:2 | Phosphatidylcholine ae C32:2 |
| PC ae C34:0 | Phosphatidylcholine ae C34:0 |
| PC ae C38:0 | Phosphatidylcholine ae C38:0 |
| C18:1 | Octadecenoylcarnitine |

TABLE 6
APOE e4−

| Metabolite | Full_Name |
|---|---|
| C12 | Dodecanoylcarnitine |
| PC aa C36:5 | Phosphatidylcholine aa C36:5 |
| Thr | Threonine |
| SM C16:1 | Sphingomyelin C16:1 |
| SM(OH) C24:1 | Hydroxysphingomyelin C24:1 |
| PC ae C42:4 | Phosphatidylcholine ae C42:4 |
| Ala | Alanine |
| PC ae C36:5 | Phosphatidylcholine ae C36:5 |
| Creatinine | Creatinine |
| PC ae C36:0 | Phosphatidylcholine ae C36:0 |
| Cit | Citrulline |
| SM C20:2 | Sphingomyelin C20:2 |
| PC aa C42:0 | Phosphatidylcholine aa C42:0 |
| PC ae C44:3 | Phosphatidylcholine ae C44:3 |
| lysoPC a C16:1 | Lysophosphatidylcholine a C16:1 |
| PC aa C40:6 | Phosphatidylcholine aa C40:6 |
| C14:1 | Tetradecenoylcarnitine |
| C4 | Butyrylcarnitine |
| PC aa C38:6 | Phosphatidylcholine aa C38:6 |
| lysoPC a C18:1 | Lysophosphatidylcholine a C18:1 |
| PC aa C32:1 | Phosphatidylcholine aa C32:1 |
| PC aa C38:0 | Phosphatidylcholine aa C38:0 |
| PC aa C36:6 | Phosphatidylcholine aa C36:6 |
| PC ae C42:2 | Phosphatidylcholine ae C42:2 |
| PC ae C40:6 | Phosphatidylcholine ae C40:6 |
| Metabolite & Clinical | Full_Name |
| AGE | AGE |
| PC ae C32:2 | Phosphatidylcholine ae C32:2 |
| PC aa C38:6 | Phosphatidylcholine aa C38:6 |
| BMI.bl | Body Mass Index |
| PC ae C36:0 | Phosphatidylcholine ae C36:0 |
| C12 | Dodecanoylcarnitine |
| SM C18:1 | Sphingomyelin C18:1 |

TABLE 7
Male APOE e4+

| Metabolite | Full_Name |
|---|---|
| Taurine | Taurine |
| PC ae C36:5 | Phosphatidylcholine ae C36:5 |
| PC aa C42:6 | Phosphatidylcholine aa C42:6 |
| PC aa C38:4 | Phosphatidylcholine aa C38:4 |
| SM(OH) C22:1 | Hydroxysphingomyelin C22:1 |
| PC ae C32:1 | Phosphatidylcholine ae C32:1 |
| PC ae C32:2 | Phosphatidylcholine ae C32:2 |
| lysoPC a C18:0 | Lysophosphatidylcholine a C18:0 |
| Asn | Asparagine |
| Metabolite & Clinic | Full_Name |
| SM(OH) C22:1 | Hydroxysphingomyelin C22:1 |
| C18 | Octadecanoylcarnitine |
| PC aa C40:3 | Phosphatidylcholine aa C40:3 |
| PC aa C38:4 | Phosphatidylcholine aa C38:4 |
| Taurine | Taurine |
| PC ae C36:5 | Phosphatidylcholine ae C36:5 |
| PC ae C40:2 | Phosphatidylcholine ae C40:2 |
| SM C24:0 | Sphingomyelin C24:0 |
| PC ae C42:5 | Phosphatidylcholine ae C42:5 |
| PTEDUCAT | PTEDUCAT |
| C14:1-OH | Hydroxytetradecenoylcarnitine |
| PC ae C36:4 | Phosphatidylcholine ae C36:4 |
| PC ae C30:0 | Phosphatidylcholine ae C30:0 |
| PC aa C36:0 | Phosphatidylcholine aa C36:0 |
| PC ae C36:0 | Phosphatidylcholine ae C36:0 |
| SM(OH) C14:1 | Hydroxysphingomyelin C14:1 |
| PC aa C42:6 | Phosphatidylcholine aa C42:6 |

TABLE 8
Male APOE e4−

| Metabolite | Full_Name |
|---|---|
| C14:1-OH | Hydroxytetradecenoylcarnitine |
| Ala | Alanine |
| PC ae C40:4 | Phosphatidylcholine ae C40:4 |
| PC ae C36:0 | Phosphatidylcholine ae C36:0 |
| SM(OH) C24:1 | Hydroxysphingomyelin C24:1 |
| PC ae C40:2 | Phosphatidylcholine ae C40:2 |
| PC ae C38:5 | Phosphatidylcholine ae C38:5 |
| Tyr | Tyrosine |
| Sarcosine | Sarcosine |
| lysoPC a C16:0 | Lysophosphatidylcholine a C16:0 |
| C9 | Nonanoylcarnitine |
| Metabolite & Clinic | Full_Name |
| Sarcosine | Sarcosine |
| AGE | AGE |
| PC aa C38:6 | Phosphatidylcholine aa C38:6 |
| alpha-AAA | alpha-Aminoadipic acid |

TABLE 8
Female APOE e4+

| Metabolite | Full_Name |
|---|---|
| PC aa C34:4 | Phosphatidylcholine aa C34:4 |
| PC ae C40:2 | Phosphatidylcholine ae C40:2 |
| lysoPC a C18:1 | Lysophosphatidylcholine a C18:1 |
| PC aa C38:3 | Phosphatidylcholine aa C38:3 |
| PC aa C42:5 | Phosphatidylcholine aa C42:5 |
| Metabolite + Clinic | Full_Name |
| PC aa C34:4 | Phosphatidylcholine aa C34:4 |
| Trp | Tryptophan |
| PC aa C40:5 | Phosphatidylcholine aa C40:5 |
| BMI.bl | Body Mass Index |
| PTEDUCAT | PTEDUCAT |
| Thr | Threonine |
| PC aa C42:5 | Phosphatidylcholine aa C42:5 |

TABLE 9
Female APOE e4−

| Metabolite | Full_Name |
|---|---|
| C16:1 | Hexadecenoylcarnitine |
| SM C16:0 | Sphingomyelin C16:0 |
| C12 | Dodecanoylcarnitine |
| C9 | Nonanoylcarnitine |
| PC ae C36:4 | Phosphatidylcholine ae C36:4 |
| Lys | Lysine |
| PC ae C32:2 | Phosphatidylcholine ae C32:2 |
| PC ae C44:6 | Phosphatidylcholine ae C44:6 |
| PC aa C36:5 | Phosphatidylcholine aa C36:5 |
| SM(OH) C14:1 | Hydroxysphingomyelin C14:1 |
| PC aa C34:3 | Phosphatidylcholine aa C34:3 |
| Creatinine | Creatinine |
| PC ae C36:0 | Phosphatidylcholine ae C36:0 |
| PC aa C28:1 | Phosphatidylcholine aa C28:1 |
| SM C26:0 | Sphingomyelin C26:0 |
| PC ae C40:3 | Phosphatidylcholine ae C40:3 |
| SM(OH) C22:2 | Hydroxysphingomyelin C22:2 |
| Metabolite + Clinic | Full_Name |
| AGE | AGE |
| PC aa C38:0 | Phosphatidylcholine aa C38:0 |
| BMI.bl | Body Mass Index |
| Trp | Tryptophan |

TABLE 10
Dietary interventions for males
Metabolite ID
L-valine
L-Isoleucine
L-leucine
L-lysine
L-Tryptophan
beta-alanine
L-arginine
Taurine
L-histidine
Creatine
L-Citrulline
L-Threonine

TABLE 11
Dietary interventions for females

| Metabolite ID | Name |
|---|---|
| Choline bitartrate | Choline bitartrate |
| L-α-Phosphatidylcholine | L-α-Phosphatidylcholine |
| Phosphotidycholine | Phosphotidycholine from egg yolk |
| PC aa C36:6 | 1-Myristoyl-2-docosahexaenoyl-sn-glycero-3-phosphocholine |
| PC aa C30:0 | 1-Myristoyl-2-(1-enyl-palmitoyl)-sn-glycero-3-phosphocholine |
| PC ae C38:0 | 1-Palmityl-2-behenoyl-sn-glycero-3-phosphocholine |
| PC ae C40:6 | PC(O-18:0/22:6(4Z,7Z,10Z,13Z,16Z,19Z)) |
| PC aa C34:4 | 1-Myristoyl-2-arachidonoyl-sn-glycero-3-phosphocholine |
| PC aa C36:5 | 1-Myristoyl-2-docosapentaenoyl-sn-glycero-3-phosphocholine |
| PC aa C38:6 | 1-Palmitoyl-2-docosahexaenoyl-sn-glycero-3-phosphocholine |
| PC ae C30:0 | [(2R)-2-hexadecanoyloxy-3-tetradecoxypropyl] 2- |
| | (trimethylazaniumyl)ethyl phosphate |
| PC aa C32:1 | 1-Myristoyl-2-oleoyl-sn-glycero-3-phosphocholine |
| PC aa C32:0 | 1-Myristoyl-2-(1-enyl-stearoyl)-sn-glycero-3-phosphocholine |
| PC aa C38:0 | 1-Behenoyl-2-(1-enyl-palmitoyl)-sn-glycero-3-phosphocholine |
| PC aa C40:6 | 1-Stearoyl-2-docosahexaenoyl-sn-glycero-3-phosphocholine |
| PC ae C40:1 | 1-(9Z-Octadecenyl)-2-docosanoyl-sn-glycero-3-phosphocholine |
| PC ae C42:3 | 1-Lignoceryl-2-g-linolenoyl-sn-glycero-3-phosphocholine |
| PC ae C42:2 | PC(o-18:2(9Z,12Z)/24:0) |
| PC aa C42:1 | 1-Erucoyl-2-arachidonyl-sn-glycero-3-phosphocholine |
| PC ae C40:5 | PC(o-20:1(11Z)/20:4(8Z,11Z,14Z,17Z)) |
| PC ae C38:6 | PC(O-16:0/22:6(4Z,7Z,10Z,13Z,16Z,19Z)) |
| PC aa C34:3 | 1-Myristoyl-2-meadoyl-sn-glycero-3-phosphocholine |
| PC aa C40:4 | 1-Arachidonoyl-2-arachidonyl-sn-glycero-3-phosphocholine |
| PC aa C38:4 | 1-Arachidonoyl-2-(1-enyl-stearoyl)-sn-glycero-3-phosphocholine |
| PC aa C38:5 | 1-Palmitoyl-2-docosapentaenoyl-sn-glycero-3-phosphocholine |
| PC ae C40:4 | 1-Eicosyl-2-(5Z,8Z,11Z,14Z-eicosatetraenoyl)-glycero-3- |
| | phosphocholine |
| L-Tryptophan | |
| L-lysine | |
| Taurine | |
| L-Tyrosine | |
| L-Histidine | |
| L-Threonine | |
| Creatine | |
| L-Citrulline | |
| beta-alanine | |
| NADH | |
| NAD+ | |

TABLE 12
Dietary interventions for male APOE e4+

| Metabolite ID | Name |
|---|---|
| Sphingomyelin | Sphingomyelin |
| SM C16:0 | N-Palmitoylsphingomyelin |
| SM C24:1 | [(E,2S,3R)-3-hydroxy-2-[[(Z)-tetracos-15- |
| | enoyl]amino]octadec-4-enyl] 2- |
| | (trimethylazaniumyl)ethyl phosphate |
| SM C24:0 | [(E,2R,3S)-3-hydroxy-2- |
| | (tetracosanoylamino)octadec-4-enyl] 2- |
| | (trimethylazaniumyl)ethyl phosphate |
| SM C16:1 | 2-[[(E,2S,3R)-2-[[(E)-Hexadec-9- |
| | enoyl]amino]-3-hydroxynonadec-11- |
| | enoxy]-hydroxyphosphoryl]oxyethyl- |
| | trimethylazanium |
| SM(OH) C14:1 | N-[(9Z)-3-Hydroxytetradec-9- |
| | enoyl]sphingosine-1-phosphocholine |
| PC ae C36:3 | 1-Vaccenyl-2-linoleoyl-sn-glycero-3- |
| | phosphocholine |
| PC aa C40:2 | 1-Stearoyl-2-docosadienoyl-sn-glycero-3- |
| | phosphocholine |
| PC ae C34:3 | [(2R)-3-[(Z)-hexadec-9-enoxy]-2-[(9Z,12Z)- |
| | octadeca-9,12-dienoyl]oxypropyl] 2- |
| | (trimethylazaniumyl)ethyl phosphate |
| PC ae C34:2 | PC(O-16:0/18:2(9Z,12Z)) |
| PC ae C34:1 | 1-Palmitoleyl-2-stearoyl-sn-glycero-3- |
| | phosphocholine |
| PC aa C42:2 | 1-Arachidonyl-2-docosadienoyl-sn- |
| | glycero-3-phosphocholine |
| PC aa C40:3 | 1-α-Linolenoyl-2-behenoyl-sn-glycero-3- |
| | phosphocholine |
| PC ae C32:1 | 1-Hexadecyl-2-(9Z-hexadecenoyl)-glycero- |
| | 3-phosphocholine |
| PC ae C30:0 | [(2R)-2-hexadecanoyloxy-3- |
| | tetradecoxypropyl] 2- |
| | (trimethylazaniumyl)ethyl phosphate |
| PC ae C36:4 | 1-Palmityl-2-eicsoate |
| PC ae C36:2 | 1-Stearyl-2-linoleoyl-sn-glycero-3- |
| | phosphocholine |
| PC ae C38:3 | 1-Arachidyl-2-g-linolenoyl-sn-glycero-3- |
| | phosphocholine |
| PC aa C32:0 | 1-Myristoyl-2-(1-enyl-stearoyl)-sn- |
| | glycero-3-phosphocholine |
| PC ae C34:0 | [(2R)-3-hexadecoxy-2- |
| | octadecanoyloxypropyl] 2- |
| | (trimethylazaniumyl)ethyl phosphate |
| PC ae C36:1 | 1-Palmitoleyl-2-arachidonyl-sn-glycero-3- |
| | phosphocholine |
| PC aa C36:1 | 1-Myristoyl-2-erucoyl-sn-glycero-3- |
| | phosphocholine |
| PC ae C32:2 | 1-[(9Z)-Hexadecenyl]-2-[(9Z)- |
| | hexadecenoyl]-sn-glycero-3- |
| | phosphocholine |
| PC ae C40:3 | 1-Behenyl-2-g-linolenoyl-sn-glycero-3- |
| | phosphocholine |
| PC aa C42:1 | 1-Erucoyl-2-arachidonyl-sn-glycero-3- |
| | phosphocholine |
| PC ae C42:3 | 1-Lignoceryl-2-g-linolenoyl-sn-glycero-3- |
| | phosphocholine |
| PC ae C42:1 | 1-Oleyl-2-lignoceroyl-sn-glycero-3- |
| | phosphocholine |
| PC ae C42:2 | PC(o-18:2(9Z,12Z)/24:0) |
| Phosphotidycholine | Phosphotidycholine from egg yolk |
| L-α-Phosphatidylcholine | L-α-Phosphatidylcholine |
| Choline bitartrate | Choline bitartrate |
| L-Taurine | L-Taurine |
| beta-alanine | beta-alanine |
| L-lysine | L-lysine |
| L-Threonine | L-Threonine |
| Creatine | Creatine |
| L-histidine | L-histidine |
| L-Citrulline | L-Citrulline |
| L-arginine | L-arginine |
| L-Tryptophan | L-Tryptophan |
| NADH | NADH |
| NAD+ | NAD+ |

TABLE 13
Dietary interventions for male APOE e4−

| Metabolite ID | CAS_number |
|---|---|
| L-valine | 72-18-4 |
| L-Isoleucine | 73-32-5 |
| L-leucine | 61-90-5 |
| L-lysine | 56-87-1 |
| L-arginine | 74-79-3 |
| L-Tyrosine | 60-18-4 |
| Creatine | 57-00-1 |
| L-Citrulline | 372-75-8 |
| Taurine | 107-35-7 |
| beta-alanine | 107-95-9 |

TABLE 14
Dietary interventions for female APOE e4+

| Metabolite ID | Name |
|---|---|
| Choline bitartrate | Choline bitartrate |
| Phosphotidycholine | Phosphotidycholine from egg yolk |
| L-α-Phosphatidylcholine | L-α-Phosphatidylcholine |
| PC aa C34:4 | 1-Myristoyl-2-arachidonoyl-sn-glycero-3-phosphocholine |
| PC ae C36:4 | 1-Palmityl-2-eicsoate |
| PC aa C30:0 | 1-Myristoyl-2-(1-enyl-palmitoyl)-sn-glycero-3-phosphocholine |
| PC aa C34:3 | 1-Myristoyl-2-meadoyl-sn-glycero-3-phosphocholine |
| PC aa C36:6 | 1-Myristoyl-2-docosahexaenoyl-sn-glycero-3-phosphocholine |
| PC aa C40:4 | 1-Arachidonoyl-2-arachidonyl-sn-glycero-3-phosphocholine |
| PC aa C38:5 | 1-Palmitoyl-2-docosapentaenoyl-sn-glycero-3-phosphocholine |
| PC ae C34:3 | [(2R)-3-[(Z)-hexadec-9-enoxy]-2-[(9Z,12Z)-octadeca-9,12- |
| | dienoyl]oxypropyl] 2-(trimethylazaniumyl)ethyl phosphate |
| PC ae C36:5 | [(2R)-3-[(Z)-hexadec-9-enoxy]-2-[(5Z,8Z,11Z,14Z)-icosa-5,8,11,14- |
| | tetraenoyl]oxypropyl] 2-(trimethylazaniumyl)ethyl phosphate |
| PC ae C36:3 | 1-Vaccenyl-2-linoleoyl-sn-glycero-3-phosphocholine |
| PC ae C38:5 | [(2R)-2-[(5Z,8Z,11Z,14Z)-icosa-5,8,11,14-tetraenoyl]oxy-3-[(Z)- |
| | octadec-9-enoxy]propyl] 2-(trimethylazaniumyl)ethyl phosphate |
| PC ae C38:4 | [(2R)-2-[(5Z,8Z,11Z,14Z)-icosa-5,8,11,14-tetraenoyl]oxy-3- |
| | octadecoxypropyl] 2-(trimethylazaniumyl)ethyl phosphate |
| PC ae C30:0 | [(2R)-2-hexadecanoyloxy-3-tetradecoxypropyl] 2- |
| | (trimethylazaniumyl)ethyl phosphate |
| PC aa C32:1 | 1-Myristoyl-2-oleoyl-sn-glycero-3-phosphocholine |
| PC aa C32:0 | 1-Myristoyl-2-(1-enyl-stearoyl)-sn-glycero-3-phosphocholine |
| PC ae C34:0 | [(2R)-3-hexadecoxy-2-octadecanoyloxypropyl] 2- |
| | (trimethylazaniumyl)ethyl phosphate |
| PC ae C38:0 | 1-Palmityl-2-behenoyl-sn-glycero-3-phosphocholine |
| PC aa C40:6 | 1-Stearoyl-2-docosahexaenoyl-sn-glycero-3-phosphocholine |
| PC aa C36:5 | 1-Myristoyl-2-docosapentaenoyl-sn-glycero-3-phosphocholine |
| PC aa C38:6 | 1-Palmitoyl-2-docosahexaenoyl-sn-glycero-3-phosphocholine |
| PC aa C38:3 | 1-Stearoyl-2-meadoyl-sn-glycero-3-phosphocholine |
| PC ae C42:1 | 1-Oleyl-2-lignoceroyl-sn-glycero-3-phosphocholine |
| PC ae C40:4 | 1-Eicosyl-2-(5Z,8Z,11Z,14Z-eicosatetraenoyl)-glycero-3- |
| | phosphocholine |
| PC aa C40:5 | PC(18:0/22:5(4Z,7Z,10Z,13Z,16Z)) |
| PC aa C38:4 | 1-Arachidonoyl-2-(1-enyl-stearoyl)-sn-glycero-3-phosphocholine |
| PC ae C40:1 | 1-(9Z-Octadecenyl)-2-docosanoyl-sn-glycero-3-phosphocholine |
| PC aa C42:5 | 1-Docosapentaenoyl-2-arachidonyl-sn-glycero-3-phosphocholine |
| PC ae C38:6 | PC(O-16:0/22:6(4Z,7Z,10Z,13Z,16Z,19Z)) |
| PC ae C32:2 | 1-[(9Z)-Hexadecenyl]-2-[(9Z)-hexadecenoyl]-sn-glycero-3- |
| | phosphocholine |
| PC ae C32:1 | 1-Hexadecyl-2-(9Z-hexadecenoyl)-glycero-3-phosphocholine |
| PC ae C34:2 | PC(O-16:0/18:2(9Z,12Z)) |
| PC ae C34:1 | 1-Palmitoleyl-2-stearoyl-sn-glycero-3-phosphocholine |
| PC aa C40:3 | 1-a-Linolenoyl-2-behenoyl-sn-glycero-3-phosphocholine |
| PC ae C36:2 | 1-Stearyl-2-linoleoyl-sn-glycero-3-phosphocholine |
| PC ae C40:5 | PC(o-20:1(11Z)/20:4(8Z,11Z,14Z,17Z)) |
| PC ae C44:5 | [(2R)-3-[(13Z,16Z)-docosa-13,16-dienoxy]-2-[(10Z,13Z,16Z)-docosa- |
| | 10,13,16-trienoyl]oxypropyl] 2-(trimethylazaniumyl)ethyl |
| | phosphate |
| Sphingomyelin | |
| SM C16:0 | N-Palmitoylsphingomyelin |
| SM(OH) C16:1 | [(E,2S,3R)-3-hydroxy-2-[(Z)-3-hydroxyhexadec-9- |
| | enoyl]amino]octadec-4-enyl] 2-(trimethylazaniumyl)ethyl |
| | phosphate |
| L-Tryptophan | |
| NADH | |
| NAD+ | |
| beta-alanine | |
| L-lysine | |
| Taurine | |
| Creatine | |
| L-Threonine | |
| L-arginine | |
| L-Proline | |

TABLE 15
Dietary interventions for female APOE e4−

| Metabolite ID | Name |
|---|---|
| SM C26:0 | N-(hexacosanoyl)-sphing-4-enine-1-phosphocholine |
| SM(OH) C22:1 | N-[(13Z)-3-Hydroxydocos-13-enoyl]sphingosine-1-phosphocholine |
| | [(E,2S,3R)-2-[[(Z)-hexacos-17-enoyl]amino]-3-hydroxyoctadec-4-enyl] 2- |
| SM C26:1 | (trimethylazaniumyl)ethyl phosphate |
| SM(OH) C24:1 | N-[(15Z)-3-Hydroxytetracos-15-enoyl]sphingosine-1-phosphocholine |
| | [(E,2R,3S)-3-hydroxy-2-(tetracosanoylamino)octadec-4-enyl] 2- |
| SM C24:0 | (trimethylazaniumyl)ethyl phosphate |
| SM(OH) C22:2 | N-[(13Z,16Z)-3-Hydroxydocosa-13,16-enoyl]sphingosine-1-phosphocholine |
| | [(E,2S,3R)-3-hydroxy-2-[[(Z)-tetracos-15-enoyl]amino]octadec-4-enyl] 2- |
| SM C24:1 | (trimethylazaniumyl)ethyl phosphate |
| SM(OH) C16:1 | N-[(9Z)-3-Hydroxyhexadec-9-enoyl]sphingosine-1-phosphocholine |
| PC ae C40:3 | 1-Behenyl-2-g-linolenoyl-sn-glycero-3-phosphocholine |
| | [(2R)-2-docosanoyloxy-3-[(9Z,12Z)-octadeca-9,12-dienoxy]propyl] 2- |
| PC ae C40:2 | (trimethylazaniumyl)ethyl phosphate |
| | [(2R)-3-[(13Z,16Z)-docosa-13,16-dienoxy]-2-[(10Z,13Z,16Z)-docosa-10,13,16- |
| PC ae C44:5 | trienoyl]oxypropyl] 2-(trimethylazaniumyl)ethyl phosphate |
| Sphingomyelin | |
| Choline bitartrate | Choline bitartrate |
| Phosphotidycholine | Phosphotidycholine from egg yolk |
| L-α- | L-α-Phosphatidylcholine |
| Phosphatidylcholine | |
| L-Tryptophan | |
| L-Citrulline | |
| L-lysine | |
| Creatine | |
| Taurine | |
| beta-alanine | |
| NADH | |
| NAD+ | |

All publications, patents and patent applications mentioned in the above specification are herein incorporated by reference in their entirety. Although the disclosure has been described in connection with specific embodiments, it should be understood that the disclosure as claimed should not be unduly limited to such specific embodiments. Indeed, various modifications and variations of the described compositions and methods of the disclosure will be apparent to those of ordinary skill in the art and are intended to be within the scope of the following claims.

1. A method of measuring a panel of biomarkers in a male subject at risk of or diagnosed with a neurodegenerative disease, comprising:
measuring the level of one or more metabolites selected from the group consisting of C18, PC aa C42:6, lysoPC a C18:0, PC ae C36:4, Kynurenine, PC ae C40:2, PC ae C42:5, PC ae, C36:5, lysoPC a C20:3, Cit, lysoPC a C16:0, SM C24:0, C16, PC ae C42:3, PC aa C32:1, Ile, C4, and Creatinine in a sample from said subject.

2. A method of measuring a panel of biomarkers in a female subject at risk of or diagnosed with a neurodegenerative disease, comprising:
measuring the level of one or more metabolites selected from the group consisting of PC ae C40:2, SM C16:0, C14:1, alpha-AAA, C4, Trp, Ile, SM C26:0, SM C16:1, PC aa C36:5, and Thr in a sample from said subject.

3. A method of measuring a panel of biomarkers in a APOE e4⁺ subject at risk of or diagnosed with a neurodegenerative disease, comprising:
measuring the level of one or more metabolites selected from the group consisting of PC ae C36:0, PC ae C42:5, PC aa C32:0, PC aa C42:6, PC ae C36:5, lysoPC a C16:0, PC aa C36:0, PC aa C32:3, PC ae C34:2, C16:1, lysoPC a C18:0, SM C18:1, PC aa C36:1, PC aa C34:4, PC ae, C36:4, C18:1, PC aa C42:5, SM (OH) C24:1, and PC aa C38:0 in a sample from said subject.

4. A method of measuring a panel of biomarkers in a APOE e4⁻ subject at risk of or diagnosed with a neurodegenerative disease, comprising:
measuring the level of one or more metabolites selected from the group consisting of C12, PC aa C36:5, Thr, SM C16:1, SM (OH) C24:1, PC ae C42:4, Ala, PC ae C36:5, Creatinine, PC ae C36:0, Cit, SM C20:2, PC aa C42:0, PC ae C44:3, lysoPC a C16:1, PC aa C40:6, C14:1, C4, PC aa C38:6, lysoPC a C18:1, PC aa C32:1, PC aa C38:0, PC aa C36:6, PC ae C42:2 and PC ae C40:6 in a sample from said subject.

5. A method of measuring a panel of biomarkers in a male APOE e4⁺ subject at risk of or diagnosed with a neurodegenerative disease, comprising:
measuring the level of one or more metabolites selected from the group consisting of Taurine, PC ae C36:5, PC aa C42:6, PC aa C38:4, SM (OH) C22:1, PC ae C32:1, PC ae C32:2, lysoPC a C18:0, and Asn in a sample from said subject.

6. A method of measuring a panel of biomarkers in a female APOE e4⁺ subject at risk of or diagnosed with a neurodegenerative disease, comprising:
measuring the level of one or more metabolites selected from the group consisting of PC aa C34:4, PC ae C40:2, lysoPC a C18:1, PC aa C38:3, and PC aa C42:5 in a sample from said subject.

7. A method of measuring a panel of biomarkers in a male APOE e4⁻ subject at risk of or diagnosed with a neurodegenerative disease, comprising:
measuring the level of one or more metabolites selected from the group consisting of C14:1-OH, Ala, PC ae C40:4, PC ae C36:0, SM (OH) C24:1, PC ae C40:2, PC ae C38:5, Tyr, Sarcosine, lysoPC a C16:0, and C9 in a sample from said subject.

8. A method of measuring a panel of biomarkers in a female APOE e4⁻ subject at risk of or diagnosed with a neurodegenerative disease, comprising:
measuring the level of one or more metabolites selected from the group consisting of C16:1, SM C16:0, C12, C9, PC ae C36:4, Lys, PC ae C32:2, PC ae C44:6, PC aa C36:5, SM (OH) C14:1, PC aa C34:3, Creatinine, PC ae C36:0, PC aa C28:1, SM C26:0, PC ae C40:3, and SM (OH) C22:2 in a sample from said subject.

9. The method of any one of the preceding claims, wherein said neurodegenerative disease is Alzheimer's disease (AD).

10. The method of any of the preceding claims, further comprising administering a test compound to said subject prior to said measuring.

11. The method of claim 10, wherein said test compound is a treatment for said neurodegenerative disease.

12. The method of claim 10 or 11, wherein said test compound is a pharmaceutical agent or a nutraceutical.

13. The method of claim 12, wherein said nutraceutical comprises one or more amino acids and/or lipids.

14. The method of claim 13, wherein said nutraceutical comprises one or more amino acids and/or lipids selected from those in Tables 10-15.

15. The method of any of the preceding claims, wherein said method is repeated one or more times.

16. The method of claim 15, wherein said method is repeated monthly, yearly, or at another interval.

17. The method of any of the preceding claims, wherein the presence of said markers is indicative of a diagnosis of a neurodegenerative disease or a prognosis for a neurodegenerative disease.

18. The method of claim 17, wherein said prognosis is a rate of cognitive decline.

19. The method of any one of the preceding claims, wherein said one or more is 2 or more.

20. The method of any one of the preceding claims, wherein said one or more is 5 or more.

21. The method of any one of the preceding claims, wherein said one or more is 10 or more.

22. The method of any one of the preceding claims, wherein said one or more is all of said metabolites.

23. The method of any one of the preceding claims, wherein said sample is whole blood or a blood product.

24. The method of claim 23, wherein said blood product is serum.

25. A method of treating or preventing cognitive decline in a subject, comprising: administering a composition comprising one or more compounds selected from those in tables 10-15 to a subject in need thereof.

26. The use of a composition comprising one or more compounds selected from those in tables 10-15 to treat or prevent cognitive decline in a subject.
